(19) United States
(12) Patent Application Publication
(10) Pub. No.: US 2005/0267156 A1
(43) Pub. Date: Dec. 1, 2005

(54) Broadspectrum 2-amino-benzothiazole sulfonamide hiv protease inhibitors

(75) Inventors: Dominique Louis Nestor Ghislain Surleraux, Machelen (BE); Piet Tom Bert Paul Wigerinck, Terhagen (BE); Daniel P. Getman, Chesterfield, MO (US)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: Tibotec Pharmaceuticals Ltd.

(21) Appl. No.: 10/523,445

(22) PCT Filed: Aug. 4, 2003
(86) PCT No.: PCT/EP03/50359

§ 371 Date: Jan. 31, 2005

Related U.S. Application Data
(60) Provisional application No. 60/427,862, filed on Nov. 20, 2002.

(30) Foreign Application Priority Data
Aug. 2, 2002 (EP) 02078231.4

(51) Int. Cl. A61K 31/4439; A61K 31/428

(52) U.S. Cl. 514/338; 514/367

(57) ABSTRACT

The present invention relates to the use of 2-amino-benzothiazoles, having the formula 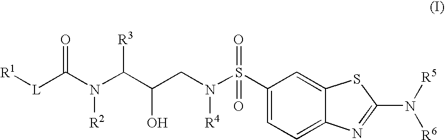
wherein R₁ is hexahydrofuro[2,3-b]furanyl, tetrahydrofuranyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino; R₂ is hydrogen or C₁₋₆alkyl; L is a direct bond, —O—, C₁₋₆alkanediyl-O— or —O—C₁₋₆alkanediyl; R₃ is phenylC₁₋₄alkyl; R₄ is C₁₋₆alkyl; R₅ is hydrogen or C₁₋₆alkyl; R₆ is hydrogen or C₁₋₆alkyl; in the manufacture of a medicament useful for inhibiting mutant HIV protease in a mammal infected with said mutant HIV protease. It also relates to novel compounds of formula (I).

This application claims priority benefit to EP Application EP 02078231.4 filed on Aug. 2, 2002 and to U.S. Provisional Application No. 60/427,862, filed on Nov. 20, 2002, the contents of which are expressly incorporated by reference herein.

The present invention relates to 2-amino-benzothiazole sulfonamides, their use as broadspectrum HIV protease inhibitors, processes for their preparation as well as pharmaceutical compositions and diagnostic kits comprising them. The present invention also concerns combinations of the present 2-aminobenzoxazole sulfonamides with another anti-retroviral agent. It further relates to their use in assays as reference compounds or as reagents.

The virus causing the acquired immunodeficiency syndrome (AIDS) is known by different names, including T-lymphocyte virus III (HTLV-III) or lymphadenopathy-associated virus (LAV) or AIDS-related virus (ARV) or human immunodeficiency virus (HIV). Up until now, two distinct families have been identified, i.e. HIV-1 and HIV-2. Hereinafter, HIV will be used to generically denote these viruses.

One of the critical pathways in a retroviral life cycle is the processing of polyprotein precursors by aspartic protease. For instance, with the HIV virus the gag-pol protein is processed by HIV protease. The correct processing of the precursor polyproteins by the aspartic protease is required for the assembly of infectious virions, thus making the aspartic protease an attractive target for antiviral therapy. In particular for HIV treatment, the HIV protease is an attractive target.

HIV protease inhibitors (PIs) are commonly administered to AIDS patients in combination with other anti-HIV compounds such as, for instance nucleoside reverse transcriptase inhibitors (NRTIs), non-nucleoside reverse transcriptase inhibitors (NNRTIs), nucleotide reverse transcriptase inhibitors (NtRTIs) or other protease inhibitors. Despite the fact that these antiretrovirals are very useful, they have a common limitation, namely, the targeted enzymes in the HIV virus are able to mutate in such a way that the known drugs become less effective, or even ineffective against these mutant HIV viruses. Or, in other words, the HIV virus creates an ever-increasing resistance against the available drugs.

Resistance of retroviruses, and in particular the HIV virus, against inhibitors is a major cause of therapy failure. For instance, half of the patients receiving anti-HIV combination therapy do not respond fully to the treatment, mainly because of resistance of the virus to one or more drugs used. Moreover, it has been shown that resistant virus is carried over to newly infected individuals, resulting in severely limited therapy options for these drug-naive patients. On the International AIDS Conference in Paris in July 2003 researchers released that the biggest study so far of resistance to AIDS drugs finds that about 10 percent of all newly infected people in Europe have drug-resistant strains. Smaller tests to determine the spread of resistance have been done in the high-risk city center of San Francisco. This test showed the highest level of resistance at 27 percent. Therefore, there is a need in the art for new compounds for retrovirus therapy, more particularly for AIDS therapy. The need in the art is particularly acute for compounds that are active not only on wild type HIV virus, but also on the increasingly more common resistant HIV viruses.

Known antiretrovirals, often administered in a combination therapy regimen, will eventually cause resistance as stated above. This often may force the physician to boost the plasma levels of the active drugs in order for said antiretrovirals to regain effectivity against the mutated HIV viruses. The consequence of which is a highly undesirable increase in pill burden. Boosting plasma levels may also lead to an increased risk of non-compliance with the prescribed therapy. Thus, it is not only important to have compounds showing activity for a wide range of HIV mutants, it is also important that there is little or no variance in the ratio between activity against mutant HIV virus and activity against wild type HIV virus (also defined as fold resistance or FR) over a broad range of mutant HIV strains. As such, a patient may remain on the same combination therapy regimen for a longer period of time since the chance that a mutant HIV virus will be sensitive to the active ingredients will be increased.

Finding compounds with a high potency on the wild type and on a wide variety of mutants is also of importance since the pill burden can be reduced if therapeutic levels are kept to a minimum. One additional way of reducing this pill burden is finding anti-HIV compounds with good bioavailability, i.e. a favorable pharmacokinetic and metabolic profile, such that the daily dose can be minimized and consequently also the number of pills to be taken.

Another favorable characteristic of an anti-HIV compound is that plasma protein binding of the inhibitor has minimal or even no effect on its potency.

Thus, there is a high medical need for protease inhibitors that are able to combat a broad spectrum of mutants of the HIV virus with little variance in fold resistance. Those protease inhibitors with a good bioavailability and little or no effect on their potency due to plasma protein binding have additional advantages.

Up until now, several protease inhibitors are on the market or are being developed. One particular core structure (depicted below) has been disclosed in a number of references, such as, WO 95/06030, WO 96/22287, WO 96/28418, WO 96/28463, WO 96/28464, WO 96/28465 and WO 97/18205. The compounds disclosed therein are described as retroviral protease inhibitors.

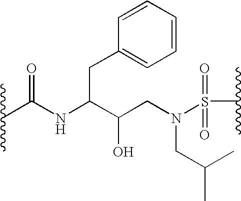

WO 99/67254 discloses 4-substituted-phenyl sulfonamides capable of inhibiting multi-drug resistant retroviral proteases.

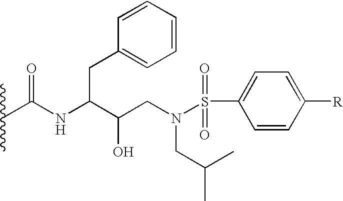

Surprisingly, the 2-amino-benzothiazole sulfonamides of the present invention are found to have a favorable virological profile. Not only are they active against wild-type HIV virus, but they also show a broadspectrum activity against various mutant HIV viruses exhibiting resistance against known protease inhibitors.

The present invention concerns the use of 2-amino-benzothiazole protease inhibitors, having the formula 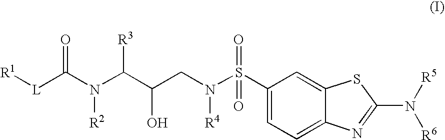
and N-oxides, salts, stereoisomeric forms, racemic mixtures, prodrugs, esters and metabolites thereof, wherein
R₁ is hexahydrofuro[2,3-b]furanyl, tetrahydrofuranyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino;
R₂ is hydrogen or C₁₋₆alkyl;
L is a direct bond, —O—, C₁₋₆alkanediyl-O— or —O—C₁₋₆alkanediyl;
R₃ is phenylC₁₋₄alkyl;
R₄ is C₁₋₆alkyl;
R₅ is hydrogen or C₁₋₆alkyl;
R₆ is hydrogen or C₁₋₆alkyl;
   in the manufacture of a medicament useful for inhibiting mutant HIV protease in a mammal infected with said mutant HIV protease. Said mammal in particular is a human being. The compounds of the present invention are in particular useful in the manufacture of a medicament useful for inhibiting a broad range of mutant HIV proteases.

A special interest goes to the free base, salt or N-oxide form of the compounds of formula (I), and their stereoisomeric forms.

Also of special interest is the use of the present compounds wherein R₁ is tetrahydrofuranyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino in the manufacture of a medicament useful for inhibiting mutant HIV protease in a mammal infected with said mutant HIV protease.

A mutant of the HIV protease enzyme is defined as an HIV protease enzyme which has at least one mutation in its amino acid sequence relative to the amino acid sequence of the wild-type HIV protease. For purposes of denoting the mutants throughout the text, the HXB2 wild type reference (HIV IIIB LAI wild type), of which the sequence can be found at NIH's GenBank, is used.

The standard of “sensitivity” or alternatively “resistance” of a HIV protease enzyme to a drug is set by the commercially available HIV protease inhibitors. As explained hereinabove, existing commercial HIV protease inhibitors may loose effectivity over time against a population of HIV virus in a patient. The reason being that under pressure of the presence of a particular HIV protease inhibitor, the existing population of HIV virus, often mainly wild type HIV protease enzyme, mutates into different mutants which may be less sensitive to that same HIV protease inhibitor. If this phenomenon occurs, one talks about resistant mutants. If those mutants are not only resistant to that one particular HIV protease inhibitor, but also to one or multiple other commercially available HIV protease inhibitors, one talks about multi-drug resistant HIV protease. One way of expressing the resistance of a mutant to a particular HIV protease inhibitor is making the ratio between the EC₅₀ of said HIV protease inhibitor against mutant HIV protease over EC₅₀ of said HIV protease inhibitor against wild type HIV protease. Said ratio is also called fold resistance (FR).

Many of the mutants occurring in the clinic have a fold resistance of 100 or more against the commercially available HIV protease inhibitors, like saquinavir, indinavir, ritonavir and nelfinavir. Clinically relevant mutants of the HIV protease enzyme can for instance be characterized by a mutation at amino acid position 10, 71 and/or 84. Examples of such clinical relevant mutant HIV proteases are listed in Table 1.

The compounds of the present invention show a fold resistance ranging between 0.01 and 100 against at least one, often against a broad range, of clinically relevant mutant HIV proteases. A particular group of compounds of formula (I) are those compounds of formula (I) showing a fold resistance against at least one mutant HIV protease ranging between 0.1 and 100, suitably ranging between 0.1 and 50, and more suitably ranging between 0.1 and 30. Of particular interest are the compounds of formula (I) showing a fold resistance against at least one mutant HIV protease ranging between 0.1 and 20, and even more interesting are those compounds of formula (I) showing a fold resistance against at least one mutant HIV protease ranging between 0.1 and 10.

Thus, the present invention relates to the use of a compound of formula (I) in the manufacture of a medicament useful for inhibiting replication of a HIV virus having a mutant HIV protease, in particular a multi-drug resistant mutant HIV protease. It also relates to the use of a compound of formula (I) in the manufacture of a medicament useful for treating or combating a disease associated with HIV viral infection wherein the protease of the HIV virus is mutant, in particular a multi-drug resistant mutant HIV protease.

In other words, the present invention relates to a method of inhibiting a mutant HIV protease, in particular a multi-drug resistant mutant HIV protease, in a mammal infected with said mutant HIV protease, said method comprising contacting said mutant HIV protease in said mammal with an effective amount of a compound of formula (I).

The present invention also relates to a method of inhibiting replication of a HIV virus, which has a mutant HIV protease, in particular a multi-drug resistant mutant HIV protease, in a mammal, said method comprising contacting said HIV virus, which has a mutant HIV protease, in said mammal with an effective amount of a compound of formula (I). The present invention further relates to a method of treating or combating a mammalian disease associated with HIV viral infection wherein the protease of the HIV virus is mutant, in particular a multi-drug resistant mutant HIV protease, said method comprising contacting said HIV virus wherein the protease of the HIV virus is mutant infecting said mammal with an effective amount of a compound of formula (I).

Of particular interest is that the compounds of the present invention can be used in the manufacture of a medicament for the treatment of individuals infected with mutant HIV protease bearing a mutation at least at one of the amino acid positions 10, 71 or 84 or at least a combination of two of these positions or at least a combination of all three.

A basic nitrogen occurring in the present compounds can be quaternized with any agent known to those of ordinary skill in the art including, for instance, lower alkyl halides, dialkyl sulfates, long chain halides and aralkyl halides.

Whenever the term “substituted” is used in defining the compounds of formula (I), it is meant to indicate that one or more hydrogens on the atom(s) indicated in the expression using “substituted” is replaced with a selection from the indicated group, provided that the indicated atom's normal valency is not exceeded, and that the substitution results in a chemically stable compound, i.e. a compound that is sufficiently robust to survive isolation to a useful degree of purity from a reaction mixture, and formulation into a therapeutic agent.

As used herein, the term “halo” or “halogen” as a group or part of a group is generic for fluoro, chloro, bromo or iodo.

The term “C₁₋₄alkyl” as a group or part of a group defines straight and branched chained saturated hydrocarbon radicals having from 1 to 4 carbon atoms, such as, for example, methyl, ethyl, propyl, butyl and 2-methyl-propyl.

The term “C₁₋₆alkyl” as a group or part of a group defines straight and branched chained saturated hydrocarbon radicals having from 1 to 6 carbon atoms such as the groups defined for C₁₋₄alkyl and pentyl, hexyl, 2-methylbutyl, 3-methylpentyl and the like.

The term “C₁₋₆alkanediyl” as a group or part of a group defines bivalent straight and branched chained saturated hydrocarbon radicals having from 1 to 6 carbon atoms such as, for example, methylene, ethan-1,2-diyl, propan-1,3-diyl, propan-1,2-diyl, butan-1,4-diyl, pentan-1,5-diyl, hexan-1,6-diyl, 2-methylbutan-1,4-diyl, 3-methylpentan-1,5-diyl and the like.

As used herein, the term “one or more” covers the possibility of all the available atoms, where appropriate, to be substituted, preferably, one, two or three.

The term “prodrug” as used throughout this text means the pharmacologically acceptable derivatives such as esters, amides and phosphates, such that the resulting in vivo biotransformation product of the derivative is the active drug as defined in the compounds of formula (I). The reference by Goodman and Gilman (The Pharmacological Basis of Therapeutics, 8^{th} ed, McGraw-Hill, Int. Ed. 1992, “Biotransformation of Drugs”, p 13-15) describing prodrugs generally is hereby incorporated. Prodrugs of a compound of the present invention are prepared by modifying functional groups present in the compound in such a way that the modifications are cleaved, either in routine manipulation or in vivo, to the parent compound. Prodrugs include compounds of the present invention wherein a hydroxy group, for instance the hydroxy group on the asymmetric carbon atom, or an amino group is bonded to any group that, when the prodrug is administered to a patient, cleaves to form a free hydroxyl or free amino, respectively.

Typical examples of prodrugs are described for instance in WO 99/33795, WO 99/33815, WO 99/33793 and WO 99/33792 all incorporated herein by reference.

Prodrugs are typically characterized by excellent aqueous solubility, increased bioavailability and are readily metabolized into the active inhibitors in vivo.

For therapeutic use, the salts of the compounds of formula (I) are those wherein the counterion is pharmaceutically or physiologically acceptable. However, salts having a pharmaceutically unacceptable counterion may also find use, for example, in the preparation or purification of a pharmaceutically acceptable compound of formula (I). All salts, whether pharmaceutically acceptable or not are included within the ambit of the present invention.

The pharmaceutically acceptable or physiologically tolerable addition salt forms which the compounds of the present invention are able to form can conveniently be prepared using the appropriate acids, such as, for example, inorganic acids such as hydrohalic acids, e.g. hydrochloric or hydrobromic acid; sulfuric; hemisulphuric, nitric; phosphoric and the like acids; or organic acids such as, for example, acetic, aspartic, dodecylsulphuric, heptanoic, hexanoic, nicotinic, propanoic, hydroxyacetic, lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-aminosalicylic, pamoic and the like acids.

Conversely said acid addition salt forms can be converted by treatment with an appropriate base into the free base form.

The compounds of formula (I) containing an acidic proton may also be converted into their non-toxic metal or amine addition salt form by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. the benzathine, N-methyl, -D-glucamine, hydrabamine salts, and salts with amino acids such as, for example, arginine, lysine and the like.

Conversely said base addition salt forms can be converted by treatment with an appropriate acid into the free acid form.

The term “salts” also comprises the hydrates and the solvent addition forms that the compounds of the present invention are able to form. Examples of such forms are e.g. hydrates, alcoholates and the like.

The N-oxide forms of the present compounds are meant to comprise the compounds of formula (I) wherein one or several nitrogen atoms are oxidized to the so-called N-oxide.

The present compounds may also exist in their tautomeric forms. Such forms, although not explicitly indicated in the above formula are intended to be included within the scope of the present invention.

The term stereochemically isomeric forms of compounds of the present invention, as used hereinbefore, defines all possible compounds made up of the same atoms bonded by the same sequence of bonds but having different three-dimensional structures which are not interchangeable, which the compounds of the present invention may possess. Unless otherwise mentioned or indicated, the chemical designation of a compound encompasses the mixture of all possible stereochemically isomeric forms that said compound might possess. Said mixture may contain all diastereomers and/or enantiomers of the basic molecular structure of said compound. All stereochemically isomeric forms of the compounds of the present invention both in pure form and in admixture with each other are intended to be embraced within the scope of the present invention.

Pure stereoisomeric forms of the compounds and intermediates as mentioned herein are defined as isomers substantially free of other enantiomeric or diastereomeric forms of the same basic molecular structure of said compounds or intermediates. In particular, the term ‘stereoisomerically pure’ concerns compounds or intermediates having a stereoisomeric excess of at least 80% (i. e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms ‘enantiomerically pure’ and ‘diastereomerically pure’ should be understood in a similar way, but then having regard to the enantiomeric excess, respectively the diastereomeric excess of the mixture in question.

Pure stereoisomeric forms of the compounds and intermediates of this invention may be obtained by the application of art-known procedures. For instance, enantiomers may be separated from each other by the selective crystallization of their diastereomeric salts with optically active acids or bases. Examples thereof are tartaric acid, dibenzoyltartaric acid, ditoluoyltartaric acid and camphosulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound would be synthesized by stereospecific methods of preparation. These methods will advantageously employ enantiomerically pure starting materials.

The diastereomeric racemates of formula (I) can be obtained separately by conventional methods. Appropriate physical separation methods that may advantageously be employed are, for example, selective crystallization and chromatography, e.g. column chromatography.

It is clear to a person skilled in the art that the compounds of formula (I) contain at least two asymmetric centers and thus may exist as different stereoisomeric forms. These two asymmetric centers are indicated with an asterisk (*) in the figure below.

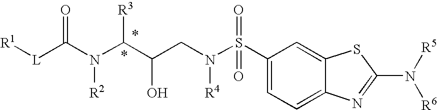

The absolute configuration of each asymmetric center that may be present in the compounds of formula (I) may be indicated by the stereochemical descriptors R and S, this R and S notation corresponding to the rules described in Pure Appl. Chem. 1976, 45, 11-30. The carbon atom bearing the hydroxy group and marked with the asterisk (*) preferably has the R configuration. The carbon atom bearing the R³ group and marked with the asterisk (*) preferably has the S configuration.

The present invention is also intended to include all isotopes of atoms occurring on the present compounds. Isotopes include those atoms having the same atomic number but different mass numbers. By way of general example and without limitation, isotopes of hydrogen include tritium and deuterium. Isotopes of carbon include C-13 and C-14.

Whenever used hereinafter, the term “compounds of formula (I)”, or “the present compounds” or similar term is meant to include the compounds of general formula (I), their N-oxides, salts, stereoisomeric forms, racemic mixtures, prodrugs, esters and metabolites, as well as their quaternized nitrogen analogues.

Some of the compounds of formula (I) have been disclosed in WO 95/06030, i.e.
{(1S,2R)-3-[(2-amino-benzothiazole-6-sulfonyl)-isobutyl-amino]-1-benzyl-2-hydroxypropyl}-carbamic acid benzyl ester;
{(1S,2R)-3-[(2-amino-benzothiazole-6-sulfonyl)-isobutyl-amino]-1-benzyl-2-hydroxypropyl}-carbamic acid pyridin-3-ylmethyl ester;
{(1S,2R)-3-[(2-amino-benzothiazole-6-sulfonyl)-isobutyl-amino]-1-benzyl-2-hydroxypropyl}-carbamic acid thiazol-5-ylmethyl ester;
{(1S,2R)-3-[(2-amino-benzothiazole-6-sulfonyl)-isobutyl-amino]-1-benzyl-2-hydroxypropyl}-2-(2,6-dimethyl-phenoxy)-acetamide;
3-amino-{(1S,2R)-3-[(2-amino-benzothiazole-6-sulfonyl)-isobutyl-amino]-1-benzyl-2-hydroxypropyl}-2-methyl-benzamide;
4-amino-{(1S,2R)-3-[(2-amino-benzothiazole-6-sulfonyl)-isobutyl-amino]-1-benzyl-2-hydroxypropyl}-2-methyl-benzamide;
5-amino-{(1S,2R)-3-[(2-amino-benzothiazole-6-sulfonyl)-isobutyl-amino]-1-benzyl-2-hydroxypropyl}-2-methyl-benzamide;
N-{(1S,2R)-3-[(2-amino-benzothiazole-6-sulfonyl)-isobutyl-amino]-1-benzyl-2-hydroxypropyl}-2-methyl-benzamide;
N-{(1S,2R)-3-[(2-amino-benzothiazole-6-sulfonyl)-isobutyl-amino]-1-benzyl-2-hydroxypropyl}-4-hydroxy-2-methyl-benzamide;
N-{(1S,2R)-3-[(2-amino-benzothiazole-6-sulfonyl)-isobutyl-amino]-1-benzyl-2-hydroxypropyl}-3-hydroxy-2-methyl-benzamide; and
{(1S,2R)-3-[(2-amino-benzothiazole-6-sulfonyl)-isobutyl-amino]-1-benzyl-2-hydroxypropyl}-carbamic acid (S)-(tetrahydrofuran-3-yl) ester.

Hence, the present invention also concerns the compounds of formula (I) 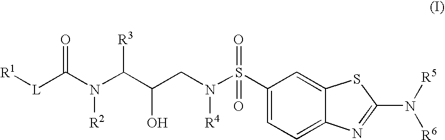
and N-oxides, salts, stereoisomeric forms, racemic mixtures, prodrugs, esters and metabolites thereof, wherein
R₁ is hexahydrofuro[2,3-b]furanyl, tetrahydrofuranyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino;
R₂ is hydrogen or C₁₋₆alkyl;
L is a direct bond, —O—, C₁₋₆alkanediyl-O— or —O—C₁₋₆alkanediyl;
R₃ is phenylC₁₋₄alkyl;
R₄ is C₁₋₆alkyl;
R₅ is hydrogen or C₁₋₆alkyl;
R₆ is hydrogen or C₁₋₆alkyl;
   provided that the compounds are other than:
{(1S,2R)-3-[(2-amino-benzothiazole-6-sulfonyl)-isobutyl-amino]-1-benzyl-2-hydroxypropyl}-carbamic acid benzyl ester;
{(1S,2R)-3-[(2-amino-benzothiazole-6-sulfonyl)-isobutyl-amino]-1-benzyl-2-hydroxypropyl}-carbamic acid pyridin-3-ylmethyl ester;
{(1S,2R)-3-[(2-amino-benzothiazole-6-sulfonyl)-isobutyl-amino]-1-benzyl-2-hydroxypropyl}-carbamic acid thiazol-5-ylmethyl ester;
{(1S,2R)-3-[(2-amino-benzothiazole-6-sulfonyl)-isobutyl-amino]-1-benzyl-2-hydroxypropyl}-2-(2,6-dimethyl-phenoxy)-acetamide;
3-amino-{(1S,2R)-3-[(2-amino-benzothiazole-6-sulfonyl)-isobutyl-amino]-1-benzyl-2-hydroxypropyl}-2-methyl-benzamide;
4-amino-{(1S,2R)-3-[(2-amino-benzothiazole-6-sulfonyl)-isobutyl-amino]-1-benzyl-2-hydroxypropyl}-2-methyl-benzamide;
5-amino-{(1S,2R)-3-[(2-amino-benzothiazole-6-sulfonyl)-isobutyl-amino]-1-benzyl-2-hydroxypropyl}-2-methyl-benzamide;
N-{(1S,2R)-3-[(2-amino-benzothiazole-6-sulfonyl)-isobutyl-amino]-1-benzyl-2-hydroxypropyl}-2-methyl-benzamide;
N-{(1S,2R)-3-[(2-amino-benzothiazole-6-sulfonyl)-isobutyl-amino]-1-benzyl-2-hydroxypropyl}-4-hydroxy-2-methyl-benzamide;
N-{(1S,2R)-3-[(2-amino-benzothiazole-6-sulfonyl)-isobutyl-amino]-1-benzyl-2-hydroxypropyl}-3-hydroxy-2-methyl-benzamide; and
{(1S,2R)-3-[(2-amino-benzothiazole-6-sulfonyl)-isobutyl-amino]-1-benzyl-2-hydroxypropyl}-carbamic acid (S)-(tetrahydrofuran-3-yl) ester.

Interesting compounds are those compounds of formula (I) wherein R¹ is hexahydrofuro[2,3-b]furanyl or oxazolyl.

Other interesting compounds are those compounds of formula (I) or those compounds belonging to any subgroup thereof wherein R₁ is hexahydrofuro[2,3-b]furanyl, tetrahydrofuranyl, oxazolyl, thiazolyl, and L is a direct bond.

Yet other interesting compounds are those compounds of formula (I) or those compounds belonging to any subgroup thereof wherein R₁ is hexahydrofuro[2,3-b]furanyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino; and L is —O—.

Still other interesting compounds are those compounds of formula (I) or those compounds belonging to any subgroup thereof wherein R₁ is hexahydrofuro[2,3-b]furanyl, tetrahydrofuranyl, oxazolyl, or phenyl substituted with one or more substituents independently selected from C₁₋₆alkyl, hydroxy, amino, halogen, aminoC₁₋₄alkyl and mono- or di(C₁₋₄alkyl)amino; and L is C₁₋₆alkanediyl-O— whereby the —O— is attached to the nitrogen of the amide.

Also interesting compounds are those compounds of formula (I) or those compounds belonging to any subgroup thereof wherein R₁ is hexahydrofuro[2,3-b]furanyl, tetrahydrofuranyl, oxazolyl, thiazolyl, pyridinyl, or phenyl optionally substituted with one or more substituents independently selected from hydroxy, amino, halogen, aminoC₁₋₄alkyl and mono- or di(C₁₋₄alkyl)amino; and L is —O—C₁₋₆alkanediyl whereby —O— is attached to the R¹ group.

A suitable group of compounds are those compounds of formula (I) or those compounds belonging to any subgroup thereof wherein at least one of R₅ and R₆ is C₁₋₆alkyl, in particular R₅ is methyl and R₆ is hydrogen or methyl, more in particular R₅ is methyl and R₆ is hydrogen.

Compounds of particular interest are those compounds of formula (I) or those compounds belonging to any subgroup thereof wherein -L-R¹ is —O-(hexahydrofuro[2,3-b]furanyl), —O-tetrahydrofuranyl, —O-methyl-(optionally substituted phenyl), —O-methyl-pyridinyl, —O-methyl-thiazolyl, —O-methyl-oxazolyl, -methyl-O-(optionally substituted phenyl) or optionally substituted phenyl. Preferably, the optional substituents on the phenyl group are methyl, amino, hydroxy, halogen, aminomethyl,

Compounds of special interest are those compounds of formula (I) or those compounds belonging to any subgroup thereof wherein R¹ is hexahydrofuro[2,3-b]furanyl, tetrahydrofuranyl, oxazolyl, thiazolyl, pyridinyl, or phenyl optionally substituted with one or more substituents independently selected from C₁₋₆alkyl, hydroxy, amino, chloro, bromo, aminoC₁₋₄alkyl and mono- or di(C₁₋₄alkyl)amino.

Suitably, one or more of the following restrictions apply to any of the above mentioned interesting subgroups of the compounds of formula (I) or subgroups of particular or special interest:
R² is hydrogen;
R³ is phenylmethyl;
R⁴ is C₁₋₄alkyl, preferably isobutyl;
R⁵ is hydrogen or methyl;
R⁶ is hydrogen or methyl.

An interesting combination for a compound of formula (I) or a compound of any subgroup thereof is formed by R² being hydrogen; R³ being phenylmethyl and R⁴ being C₁₋₄alkyl, preferably isobutyl;

A special subgroup of the compounds of formula (I) is defined as encompassing those compounds of formula (I) wherein R⁵ and R⁶ are both hydrogen.

Another special subgroup of the compounds of formula (I) or of the compounds belonging to any subgroup thereof are those compounds wherein -L-R¹ is —O-(hexahydrofuro[2,3-b]furanyl), —O-tetrahydrofuranyl, —O-methyl-thiazolyl, —O-methyl-oxazolyl, -methyl-O-(2,6-dimethylphenyl), -methyl-O-(4-aminomethyl-2,6-dimethylphenyl), -methyl-O-(4-amino-2,6-dimethylphenyl), 3-hydroxy-2-methyl-phenyl or 3-amino-2-methyl-phenyl; and R⁵ is methyl or hydrogen and R⁶ is hydrogen.

Preferred compounds are
{3-[(2-amino-benzothiazole-6-sulfonyl)-isobutyl-amino]-1-benzyl-2-hydroxypropyl}-carbamic acid hexahydro-furo[2,3-b]furan-3-yl ester;
{3-[(2-amino-benzothiazole-6-sulfonyl)-isobutyl-amino]-1-benzyl-2-hydroxypropyl}-carbamic acid thiazol-5-ylmethyl ester;
{1-benzyl-2-hydroxy-3-[isobutyl-(2-methylamino-benzothiazole-6-sulfonyl)-amino]-propyl}-carbamic acid hexahydro-furo[2,3-b]furan-3-yl ester;
{1-benzyl-3-[(2-dimethylamino-benzothiazole-6-sulfonyl)-isobutyl-amino]-2-hydroxypropyl}-carbamic acid hexahydro-furo[2,3-b]furan-3-yl ester;
{3-[(2-amino-benzothiazole-6-sulfonyl)-isobutyl-amino]-1-benzyl-2-hydroxypropyl}-carbamic acid benzyl ester;
N-{3-[(2-amino-benzothiazole-6-sulfonyl)-isobutyl-amino]-1-benzyl-2-hydroxypropyl}-2-(2,6-dimethyl-phenoxy)-acetamide;
{3-[(2-amino-benzothiazole-6-sulfonyl)-isobutyl-amino]-1-benzyl-2-hydroxypropyl}-carbamic acid pyridin-3-ylmethyl ester;
3-amino-N-{3-[(2-amino-benzothiazole-6-sulfonyl)-isobutyl-amino]-1-benzyl-2-hydroxypropyl}-2-methyl-benzamide;
N-{3-[(2-amino-benzothiazole-6-sulfonyl)-isobutyl-amino]-1-benzyl-2-hydroxypropyl}-3-hydroxy-2-methyl-benzamide;
{3-[(2-amino-benzothiazole-6-sulfonyl)-isobutyl-amino]-1-benzyl-2-hydroxypropyl}-carbamic acid tetrahydro-furan-3-yl ester;
N-{3-[(2-amino-benzothiazole-6-sulfonyl)-isobutyl-amino]-1-benzyl-2-hydroxypropyl}-2-methyl-benzamide;
N-{1-benzyl-2-hydroxy-3-[isobutyl-(2-methylamino-benzothiazole-6-sulfonyl)-amino]-propyl}-2-(2,6-dimethyl-phenoxy)-acetamide;
N-{3-[(2-amino-benzothiazole-6-sulfonyl)-isobutyl-amino]-1-benzyl-2-hydroxypropyl}-3-fluoro-2-methyl-benzamide;
N-{3-[(2-amino-benzothiazole-6-sulfonyl)-isobutyl-amino]-1-benzyl-2-hydroxypropyl}-2-(4-aminomethyl-2,6-dimethyl-phenoxy)-acetamide;
{1-benzyl-2-hydroxy-3-[isobutyl-(2-methylamino-benzothiazole-6-sulfonyl)-amino]-propyl}-carbamic acid thiazol-5-ylmethyl ester;
3-amino-N-{1-benzyl-2-hydroxy-3-[isobutyl-(2-methylamino-benzothiazole-6-sulfonyl)-amino]-propyl}-2-methyl-benzamide;
{1-benzyl-2-hydroxy-3-[isobutyl-(2-methylamino-benzothiazole-6-sulfonyl)-amino]-propyl}-carbamic acid tetrahydro-furan-3-yl ester;
N-{1-benzyl-2-hydroxy-3-[isobutyl-(2-methylamino-benzothiazole-6-sulfonyl)-amino]-propyl}-3-hydroxy-2-methyl-benzamide;
N-{3-[(2-amino-benzothiazole-6-sulfonyl)-isobutyl-amino]-1-benzyl-2-hydroxypropyl}-2-(4-iodo-2,6-dimethyl-phenoxy)-acetamide;
2-(4-aminomethyl-2,6-dimethyl-phenoxy)-N-{1-benzyl-2-hydroxy-3-[isobutyl-(2-methylamino-benzothiazole-6-sulfonyl)-amino]-propyl}-acetamide;
2-(4-amino-2,6-dimethyl-phenoxy)-N-{1-benzyl-2-hydroxy-3-[isobutyl-(2-methylamino-benzothiazole-6-sulfonyl)-amino]-propyl}-acetamide;
N-{1-benzyl-2-hydroxy-3-[isobutyl-(2-methylamino-benzothiazole-6-sulfonyl)-amino]-propyl}-4-bromo-2-methyl-benzamide;
{1-benzyl-2-hydroxy-3-[isobutyl-(2-methylamino-benzothiazole-6-sulfonyl)-amino]-propyl}-carbamic acid oxazol-5-ylmethyl ester;
4-amino-N-{1-benzyl-2-hydroxy-3-[isobutyl-(2-methylamino-benzothiazole-6-sulfonyl)-amino]-propyl}-3-hydroxy-2-methyl-benzamide; their N-oxides and salts and the stereoisomeric forms thereof.

Another group of interest includes
{1-benzyl-2-hydroxy-3-[isobutyl-(2-methylamino-benzothiazole-6-sulfonyl)-amino]-propyl}-carbamic acid hexahydro-furo[2,3-b]furan-3-yl ester;
{1-benzyl-3-[(2-dimethylamino-benzothiazole-6-sulfonyl)-isobutyl-amino]-2-hydroxypropyl}-carbamic acid hexahydro-furo[2,3-b]furan-3-yl ester;
N-{1-benzyl-2-hydroxy-3-[isobutyl-(2-methylamino-benzothiazole-6-sulfonyl)-amino]-propyl}-2-(2,6-dimethyl-phenoxy)-acetamide;
N-{3-[(2-amino-benzothiazole-6-sulfonyl)-isobutyl-amino]-1-benzyl-2-hydroxypropyl}-3-fluoro-2-methyl-benzamide;
N-{3-[(2-amino-benzothiazole-6-sulfonyl)-isobutyl-amino]-1-benzyl-2-hydroxypropyl}-2-(4-aminomethyl-2,6-dimethyl-phenoxy)-acetamide;
{1-benzyl-2-hydroxy-3-[isobutyl-(2-methylamino-benzothiazole-6-sulfonyl)-amino]-propyl}-carbamic acid thiazol-5-ylmethyl ester;
3-amino-N-{1-benzyl-2-hydroxy-3-[isobutyl-(2-methylamino-benzothiazole-6-sulfonyl)-amino]-propyl}-2-methyl-benzamide;
{1-benzyl-2-hydroxy-3-[isobutyl-(2-methylamino-benzothiazole-6-sulfonyl)-amino]-propyl}-carbamic acid tetrahydro-furan-3-yl ester;
N-{1-benzyl-2-hydroxy-3-[isobutyl-(2-methylamino-benzothiazole-6-sulfonyl)-amino]-propyl}-3-hydroxy-2-methyl-benzamide;
N-{3-[(2-amino-benzothiazole-6-sulfonyl)-isobutyl-amino]-1-benzyl-2-hydroxypropyl}-2-(4-iodo-2,6-dimethyl-phenoxy)-acetamide;
2-(4-aminomethyl-2,6-dimethyl-phenoxy)-N-{1-benzyl-2-hydroxy-3-[isobutyl-(2-methylamino-benzothiazole-6-sulfonyl)-amino]-propyl}-acetamide;
2-(4-amino-2,6-dimethyl-phenoxy)-N-{1-benzyl-2-hydroxy-3-[isobutyl-(2-methylamino-benzothiazole-6-sulfonyl)-amino]-propyl}-acetamide;
N-{1-benzyl-2-hydroxy-3-[isobutyl-(2-methylamino-benzothiazole-6-sulfonyl)-amino]-propyl}-4-bromo-2-methyl-benzamide;
{1-benzyl-2-hydroxy-3-[isobutyl-(2-methylamino-benzothiazole-6-sulfonyl)-amino]-propyl}-carbamic acid oxazol-5-ylmethyl ester;
4-amino-N-{1-benzyl-2-hydroxy-3-[isobutyl-(2-methylamino-benzothiazole-6-sulfonyl)-amino]-propyl}-3-hydroxy-2-methyl-benzamide; their N-oxides and salts and the stereoisomeric forms thereof.

Most preferred compounds are those enantiomeric forms of the compounds of formula (I) or of the compounds belonging to any subgroup thereof having a (1S,2R)-1-benzyl-2-hydroxy-propyl configuration.

Those compounds of formula (I) or those compounds belonging to any subgroup thereof in a hexahydro-furo[2,3-b]furan-3-yl ester form of the carbamic acid derivative occur preferably in a (3R,3aS,6aR) form such as, for instance, {(1S,2R)-3-[(2-amino-benzothiazole-6-sulfonyl)-isobutyl-amino]-1-benzyl-2-hydroxypropyl}-carbamic acid 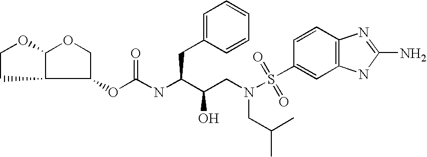
(3R,3aS,6aR)-hexahydro-furo[2,3-b]furan-3-yl ester

The compounds of formula (I) can generally be prepared using procedures analogous to those procedures described in WO 95/06030, WO 96/22287, WO 96/28418, WO 96/28463, WO 96/28464, WO 96/28465 and WO 97/18205.

Particular reaction procedures to make the present compounds are described below. In the preparations described below, the reaction products may be isolated from the medium and, if necessary, further purified according to methodologies generally known in the art such as, for example, extraction, crystallization, trituration and chromatography.

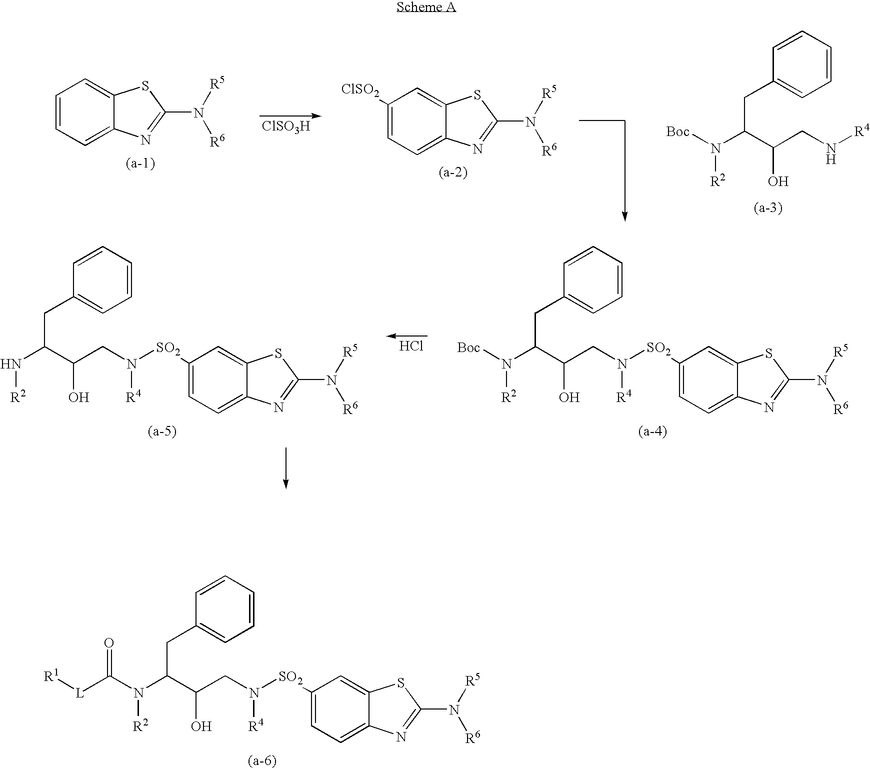

The 2-amino-6-chlorosulfonylbenzothiazole derivative (intermediate a-2) was prepared following the procedure described in EP-A-0,445,926. Intermediates a-4 were prepared by reacting an intermediate a-3, prepared according to the procedure described in WO97/18205 and also depicted in scheme B, with an intermediate a-2 in a reaction-inert solvent such as dichloromethane, and in the presence of a base such as triethylamine and at low temperature, for example at 0° C. The Boc group in the intermediate a-3 is a protective tert-butyloxycarbonyl group. Another suitable protective group such as phtalimido or benzyloxycarbonyl may conveniently replace it. Intermediates a-4 may be deprotected with an acid such as hydrochloric acid in isopropanol or with trifluoroacetic acid depending on the nature of the amino group in the 2 position of benzoxazole, in a suitable solvent such as a mixture of ethanol and dioxane, thus preparing an intermediate a-5. Said intermediate a-5 may be further reacted with an intermediate of formula R₁-L-C(═O)—OH in the presence of a base such as triethylamine (for alcohols to generate a carbamate) and optionally in the presence of 1-(3-dimethylaminopropyl)-3-ethylcarbodiimide hydrochloric acid (EDC) and 1-hydroxybenzotriazole (HOBT) (for carboxylic acids to generate an amide) or an alcohol such as tert-butanol, and in a suitable solvent such as dichloromethane; thus forming intermediates a-6.

A convenient way of preparing compounds of formula (I) wherein both R₅ and R₆ are hydrogen can be prepared analogously to the procedure described in scheme A, and whereby one of R₅ or R₆ is replaced by a suitable protective group such as, for example, an acetyl or an alkyloxycarbonyl group. In such a case, deprotection may occur simultaneously with the deprotection of the nitrogen atom on the left-hand side of the molecule.

A number of intermediates and starting materials used in the foregoing preparations are known compounds, while others may be prepared according to art-known methodologies of preparing said or similar compounds.

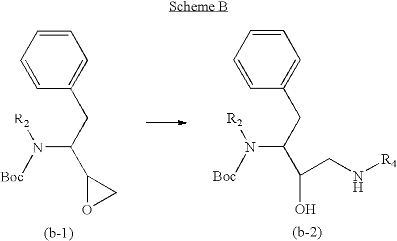

Intermediate b-2, corresponding to intermediate a-3 in scheme A, may be prepared by adding an amine of formula H₂N—R₄ to an intermediate b-1 in a suitable solvent such as isopropanol.

In scheme B, enantiomerically pure compounds of formula b-2 are only obtained if b-1 is enantiomerically pure. If b-1 is a mixture of stereoisomers, than b-2 will also consist of a mixture of stereoisomers.

The compounds of formula (I) may also be converted to the corresponding N-oxide forms following art-known procedures for converting trivalent nitrogen into its N-oxide form. Said N-oxidation reaction may generally be carried out by reacting the starting material of formula (I) with appropriate organic or inorganic peroxide. Appropriate inorganic peroxides comprise, for example, hydrogen peroxide, alkali metal or earth alkaline metal peroxides, e.g. sodium peroxide, potassium peroxide; appropriate organic peroxides may comprise peroxy acids such as, for example, benzenecarboperoxoic acid or halo substituted benzenecarboperoxoic acid, e.g. 3-chloro-benzenecarboperoxoic acid, peroxoalkanoic acids, e.g. peroxoacetic acid, alkylhydroperoxides, e.g. tert-butyl hydroperoxide. Suitable solvents are, for example, water, lower alkanols, e.g. ethanol and the like, hydrocarbons, e.g. toluene, ketones, e.g. 2-butanone, halogenated hydrocarbons, e.g. dichloromethane, and mixtures of such solvents.

The present compounds can thus be used in animals, preferably in mammals, and in particular in humans as pharmaceuticals per se, in mixtures with one another or in the form of pharmaceutical preparations.

Furthermore, the present invention relates to pharmaceutical preparations that as active constituents contain an effective dose of at least one of the compounds of formula (I) in addition to customary pharmaceutically innocuous excipients and auxiliaries. The pharmaceutical preparations normally contain 0.1 to 90% by weight of a compound of formula (I). The pharmaceutical preparations can be prepared in a manner known per se to one of skill in the art. For this purpose, at least one of a compound of formula (I), together with one or more solid or liquid pharmaceutical excipients and/or auxiliaries and, if desired, in combination with other pharmaceutical active compounds, are brought into a suitable administration form or dosage form which can then be used as a pharmaceutical in human medicine or veterinary medicine.

Pharmaceuticals which contain a compound according to the invention can be administered orally, parenterally, e.g., intravenously, rectally, by inhalation, or topically, the preferred administration being dependent on the individual case, e.g., the particular course of the disorder to be treated. Oral administration is preferred.

The person skilled in the art is familiar on the basis of his expert knowledge with the auxiliaries that are suitable for the desired pharmaceutical formulation. Beside solvents, gel-forming agents, suppository bases, tablet auxiliaries and other active compound carriers, antioxidants, dispersants, emulsifiers, antifoams, flavor corrigents, preservatives, solubilizers, agents for achieving a depot effect, buffer substances or colorants are also useful.

Due to their favorable pharmacological properties, particularly their activity against multi-drug resistant HIV protease enzymes, the compounds of the present invention are useful in the treatment of individuals infected by HIV and for the prophylaxis of these individuals. In general, the compounds of the present invention may be useful in the treatment of warm-blooded animals infected with viruses whose existence is mediated by, or depends upon, the protease enzyme. Conditions which may be prevented or treated with the compounds of the present invention, especially conditions associated with HIV and other pathogenic retroviruses, include AIDS, AIDS-related complex (ARC), progressive generalized lymphadenopathy (PGL), as well as chronic CNS diseases caused by retroviruses, such as, for example HIV mediated dementia and multiple sclerosis.

Said method of treatment comprises the systemic administration to HIV-infected subjects of an amount effective to combat the conditions associated with HIV virus with multi-drug resistant protease enzyme.

The compounds of the present invention may also find use in inhibiting ex vivo samples containing multi-drug resistant HIV-protease or expected to be exposed to multi-drug resistant HIV-protease. Hence, the present compounds may be used to inhibit multi-drug resistant HIV-protease present in a body fluid sample that contains or is suspected to contain or be exposed to multi-drug resistant HIV-protease.

Also, the combination of an antiretroviral compound and a compound of the present invention can be used as a medicine. Thus, the present invention also relates to a product containing (a) a compound of the present invention, and (b) another antiretroviral compound, as a combined preparation for simultaneous, separate or sequential use in treatment of retroviral infections, in particular, in the treatment of infections with multi-drug resistant HIV proteases. Thus, to combat or treat infections with multi-drug resistant HIV protease, or the infection and disease associated with such infections, such as Acquired Immunodeficiency Syndrome (AIDS) or AIDS Related Complex (ARC), the compounds of this invention may be co-administered in combination with for instance, binding inhibitors, such as, for example, dextran sulfate, suramine, polyanions, soluble CD4, PRO-542, BMS-806; fusion inhibitors, such as, for example, T20, T1249, 5-helix, D-peptide ADS-J1; co-receptor binding inhibitors, such as, for example, AMD 3100, AMD-3465, AMD7049, AMD3451 (Bicyclams), TAK 779; SHC-C (SCH351125), SHC-D, PRO-140RT inhibitors, such as, for example, foscarnet and prodrugs; nucleoside RTIs, such as, for example, AZT, 3TC, DDC, DDI, D4T, Abacavir, FTC, DAPD, dOTC, DPC 817; nucleotide RTIs, such as, for example, PMEA, PMPA (tenofovir); NNRTIs, such as, for example, nevirapine, delavirdine, efavirenz, 8 and 9-Cl TIBO (tivirapine), loviride, TMC-125, dapivirine, MKC-442, UC 781, UC 782, Capravirine, DPC 961, DPC963, DPC082, DPC083, calanolide A, SJ-1366, TSAO, 4″-deaminated TSAO, MV150, MV026048; RNAse H inhibitors, such as, for example, SP1093V, PD126338; TAT inhibitors, such as, for example, RO-5-3335, K12, K37; integrase inhibitors, such as, for example, L 708906, L 731988, S-1360; protease inhibitors, such as, for example, amprenavir and prodrug GW908, ritonavir, nelfinavir, saquinavir, indinavir, lopinavir, palinavir, BMS 186316, atazanavir, DPC 681, DPC 684, tipranavir, AG1776, mozenavir, GS3333, KNI-413, KNI-272, L754394, L756425, LG-71350, PD161374, PD173606, PD177298, PD178390, PD178392, PNU 140135, TMC114 maslinic acid, U-140690; glycosylation inhibitors, such as, for example, castanospermine, deoxynojirimycine.

The combination may in some cases provide a synergistic effect, whereby viral infectivity and its associated symptoms may be prevented, substantially reduced, or eliminated completely.

The compounds of the present invention may also be administered in combination with immunomodulators (e.g., bropirimine, anti-human alpha interferon antibody, IL-2, methionine enkephalin, interferon alpha, HE-2000 and naltrexone) with antibiotics (e.g., pentamidine isothiorate) cytokines (e.g. Th2), modulators of cytokines, chemokines or the receptors thereof (e.g. CCR5) or hormones (e.g. growth hormone) to ameliorate, combat, or eliminate HIV infection and its symptoms. Such combination therapy in different formulations may be administered simultaneously, separately or sequentially. Alternatively, such combination may be administered as a single formulation, whereby the active ingredients are released from the formulation simultaneously or separately.

The compounds of the present invention may also be administered in combination with modulators of the metabolization following application of the drug to an individual. These modulators include compounds that interfere with the metabolization at cytochromes, such as cytochrome P450. Some modulators inhibit cytochrome P450. It is known that several isoenzymes exist of cytochrome P450, one of which is cytochrome P450 3A4. Ritonavir is an example of a modulator of metabolization via cytochrome P450. Such combination therapy in different formulations may be administered simultaneously, separately or sequentially. Alternatively, such combination may be administered as a single formulation, whereby the active ingredients are released from the formulation simultaneously or separately. Such modulator may be administered at the same or different ratio as the compound of the present invention. Preferably, the weight ratio of such modulator vis-à-vis the compound of the present invention (modulator:compound of the present invention) is 1:1 or lower, more preferable the ratio is 1:3 or lower, suitably the ratio is 1:10 or lower, more suitably the ratio is 1:30 or lower.

For an oral administration form, compounds of the present invention are mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, cornstarch. In this case the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms.

For subcutaneous or intravenous administration, the active compounds, if desired with the substances customary therefore such as solubilizers, emulsifiers or further auxiliaries, are brought into solution, suspension, or emulsion. The compounds of formula (I) can also be lyophilized and the lyophilizates obtained used, for example, for the production of injection or infusion preparations. Suitable solvents are, for example, water, physiological saline solution or alcohols, e.g. ethanol, propanol, glycerol, in addition also sugar solutions such as glucose or mannitol solutions, or alternatively mixtures of the various solvents mentioned.

Suitable pharmaceutical formulations for administration in the form of aerosols or sprays are, for example, solutions, suspensions or emulsions of the compounds of formula (I) or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Such a preparation customarily contains the active compound in a concentration from approximately 0.1 to 50%, in particular from approximately 0.3 to 3% by weight.

In order to enhance the solubility and/or the stability of the compounds of formula (I) in pharmaceutical compositions, it can be advantageous to employ α-, β- or γ-cyclodextrins or their derivatives. Also co-solvents such as alcohols may improve the solubility and/or the stability of the compounds of formula (I) in pharmaceutical compositions. In the preparation of aqueous compositions, addition salts of the subject compounds are obviously more suitable due to their increased water solubility.

Appropriate cyclodextrins are α-, β- or γ-cyclodextrins (CDs) or ethers and mixed ethers thereof wherein one or more of the hydroxy groups of the anhydroglucose units of the cyclodextrin are substituted with C₁₋₆alkyl, particularly methyl, ethyl or isopropyl, e.g. randomly methylated β-CD; hydroxyC₁₋₆alkyl, particularly hydroxyethyl, hydroxypropyl or hydroxybutyl; carboxyC₁₋₆alkyl, particularly carboxymethyl or carboxyethyl; C₁₋₆alkyl-carbonyl, particularly acetyl; C₁₋₆alkyloxycarbonylC₁₋₆alkyl or carboxyC₁₋₆alkyloxyC₁₋₆alkyl, particularly carboxymethoxypropyl or carboxyethoxypropyl; C₁₋₆alkylcarbonyloxyC₁₋₆alkyl, particularly 2-acetyloxypropyl. Especially noteworthy as complexants and/or solubilizers are β-CD, randomly methylated β-CD, 2,6-dimethyl-β-CD, 2-hydroxyethyl-β-CD, 2-hydroxyethyl-γ-CD, 2-hydroxy-propyl-γ-CD and (2-carboxymethoxy)propyl-β-CD, and in particular 2-hydroxy-propyl-β-CD (2-HP-β-CD).

The term mixed ether denotes cyclodextrin derivatives wherein at least two cyclodextrin hydroxy groups are etherified with different groups such as, for example, hydroxy-propyl and hydroxyethyl.

An interesting way of formulating the present compounds in combination with a cyclodextrin or a derivative thereof has been described in EP-A-721,331. Although the formulations described therein are with antifungal active ingredients, they are equally interesting for formulating the compounds of the present invention. The formulations described therein are particularly suitable for oral administration and comprise an antifungal as active ingredient, a sufficient amount of a cyclodextrin or a derivative thereof as a solubilizer, an aqueous acidic medium as bulk liquid carrier and an alcoholic co-solvent that greatly simplifies the preparation of the composition. Said formulations may also be rendered more palatable by adding pharmaceutically acceptable sweeteners and/or flavors.

Other convenient ways to enhance the solubility of the compounds of the present invention in pharmaceutical compositions are described in WO 94/05263, WO 98/42318, EP-A-499,299 and WO 97/44014, all incorporated herein by reference.

More in particular, the present compounds may be formulated in a pharmaceutical composition comprising a therapeutically effective amount of particles consisting of a solid dispersion comprising (a) a compound of formula (I), and (b) one or more pharmaceutically acceptable water-soluble polymers.

The term “a solid dispersion” defines a system in a solid state (as opposed to a liquid or gaseous state) comprising at least two components, wherein one component is dispersed more or less evenly throughout the other component or components. When said dispersion of the components is such that the system is chemically and physically uniform or homogenous throughout or consists of one phase as defined in thermodynamics, such a solid dispersion is referred to as “a solid solution”. Solid solutions are preferred physical systems because the components therein are usually readily bioavailable to the organisms to which they are administered.

The term “a solid dispersion” also comprises dispersions that are less homogenous throughout than solid solutions. Such dispersions are not chemically and physically uniform throughout or comprise more than one phase.

The water-soluble polymer in the particles is conveniently a polymer that has an apparent viscosity of 1 to 100 mPa.s when dissolved in a 2% aqueous solution at 20° C. solution.

Preferred water-soluble polymers are hydroxypropyl methylcelluloses or HPMC. HPMC having a methoxy degree of substitution from about 0.8 to about 2.5 and a hydroxypropyl molar substitution from about 0.05 to about 3.0 is generally water soluble. Methoxy degree of substitution refers to the average number of methyl ether groups present per anhydroglucose unit of the cellulose molecule. Hydroxy-propyl molar substitution refers to the average number of moles of propylene oxide that have reacted with each anhydroglucose unit of the cellulose molecule.

First preparing a solid dispersion of the components, and then optionally grinding or milling that dispersion allows one to prepare the particles as defined hereinabove. Various techniques exist for preparing solid dispersions including melt-extrusion, spray-drying and solution-evaporation, melt-extrusion being preferred.

It may further be convenient to formulate the present compounds in the form of nanoparticles which have a surface modifier adsorbed on the surface thereof in an amount sufficient to maintain an effective average particle size of less than 1000 nm. Useful surface modifiers are believed to include those that physically adhere to the surface of the antiretroviral agent but do not chemically bond to the antiretroviral agent.

Suitable surface modifiers can preferably be selected from known organic and inorganic pharmaceutical excipients. Such excipients include various polymers, low molecular weight oligomers, natural products and surfactants. Preferred surface modifiers include nonionic and anionic surfactants.

Yet another interesting way of formulating the present compounds involves a pharmaceutical composition whereby the present compounds are incorporated in hydrophilic polymers and applying this mixture as a coat film over many small beads, thus yielding a composition with good bioavailability which can conveniently be manufactured and which is suitable for preparing pharmaceutical dosage forms for oral administration.

Said beads comprise (a) a central, rounded or spherical core, (b) a coating film of a hydrophilic polymer and an antiretroviral agent and (c) a seal-coating polymer layer.

Materials suitable for use as cores in the beads are manifold, provided that said materials are pharmaceutically acceptable and have appropriate dimensions and firmness. Examples of such materials are polymers, inorganic substances, organic substances, and saccharides and derivatives thereof.

The route of administration may depend on the condition of the subject, co-medication and the like.

Another aspect of the present invention concerns a kit or container comprising a compound of formula (I) in an amount effective for use as a standard or reagent in a test or assay for determining the ability of a potential pharmaceutical to inhibit multi-drug resistant HIV protease, HIV growth, or both. This aspect of the invention may find its use in pharmaceutical research programs.

The compounds of the present invention can be used in phenotypic resistance monitoring assays, such as known recombinant assays, in the clinical management of resistance developing diseases such as HIV. A particularly useful resistance monitoring system is a recombinant assay known as the Antivirogram™. The Antivirogram™ is a highly automated, high throughput, second generation, recombinant assay that can measure susceptibility, especially viral susceptibility, to the compounds of the present invention. (Hertogs K, de Bethune M P, Miller V et al. Antimicrob Agents Chemother, 1998; 42(2):269-276, incorporated by reference).

Interestingly, the compounds of the present invention may comprise chemically reactive moieties capable of forming covalent bonds to localized sites such that said compound have increased tissue retention and half-lives. The term “chemically reactive group” as used herein refers to chemical groups capable of forming a covalent bond. Reactive groups will generally be stable in an aqueous environment and will usually be carboxy, phosphoryl, or convenient acyl group, either as an ester or a mixed anhydride, or an imidate, or a maleimidate thereby capable of forming a covalent bond with functionalities such as an amino group, a hydroxy or a thiol at the target site on for example blood components such as albumine. The compounds of the present invention may be linked to maleimide or derivatives thereof to form conjugates.

The dose of the present compounds or of the physiologically tolerable salt(s) thereof to be administered depends on the individual case and, as customary, is to be adapted to the conditions of the individual case for an optimum effect. Thus it depends, of course, on the frequency of administration and on the potency and duration of action of the compounds employed in each case for therapy or prophylaxis, but also on the nature and severity of the infection and symptoms, and on the sex, age, weight, co-medication, and individual responsiveness of the human or animal to be treated and on whether the therapy is acute or prophylactic. Customarily, the daily dose of a compound of formula (I) in the case of administration to a patient approximately 75 kg in weight is 1 mg to 3 g, suitably 1 mg to 1 g, preferably 3 mg to 0.5 g, more preferably 5 mg to 300 mg. The dose can be administered in the form of an individual dose, or divided into several, e.g. two, three, or four, individual doses.

Experimental Part

Preparation of the Compounds of Formula (I)

The nomenclature used throughout the description is based on Chemical Abstracts Services Nomenclature.

### EXAMPLE 1

### Compound 2

To a mixture of 825 mg 2-amino-benzothiazole-6-sulfonic acid (3-amino-2-hydroxy-4-phenyl-butyl)-isobutyl amide and 373 mg triethylamine in dichloromethane was added 452 mg 1-[[[[(3R,3aS,6aR)-hexahydrofuro[2,3-b]furan-3-yl]oxy]carbonyl]oxy]-2,5-pyrrolidine-dione (described in W09967417). This mixture was stirred at room temperature for 12 hours. After evaporation of dichloromethane under reduced pressure, the crude product was purified on silica, yielding 270 mg 24.8% compound 2.

### EXAMPLE 2

### Compound 4

To a mixture of 350 mg 2-methylamino-benzothiazole-6-sulfonic acid (3-amino-2-hydroxy-4-phenyl-butyl)-isobutyl amide and 200 mg triethylamine in dichloromethane was added 210 mg 1-[[[[hexahydrofuro[2,3-b]furan-3-yl]oxy]carbonyl]oxy]-2,5-pyrrolidinedione (described in W09967417). This mixture was stirred at room temperature for 12 hours. After evaporation of dichloromethane under reduced pressure, the crude product was purified on silica, yielding 260 mg (55%) of compound 4.

### EXAMPLE 3

### Compound 6

To a mixture of 420 mg 2-dimethylamino-benzothiazole-6-sulfonic acid (3-amino-2-hydroxy-4-phenyl-butyl)-isobutyl amide and 98 mg triethylamine in dichloromethane was added 230 mg 1-[[[[hexahydrofuro[2,3-b]furan-3-yl]oxy]carbonyl]oxy]-2,5-pyrrolidinedione (described in W09967417). This mixture was stirred at room temperature for 12 hours. After evaporation of dichloromethane under reduced pressure, the crude product was purified on silica, yielding 500 mg 90% of compound 6.

### EXAMPLE 4

### Compound 17

A mixture of 800 mg of 2-amino-benzothiazole-6-sulfonic acid (3-amino-2-hydroxy-4-phenyl-butyl)-isobutyl amide, 50 mg of HOBT (hydroxybenzotriazol), 420 mg of EDC and 668 mg of (3,4,5-trimethyl-benzyl)-carbamic acid tert-butyl ester compound with hydroxy acetic acid in 80 ml of dichloromethane, was stirred overnight at room temperature. The reaction mixture was then washed with water and brine. The organic layer was separated, dried and the solvent evaporated. The residue was purified by column chromatography, yielding 1 g (75%) of [4-({3-[(2-amino-benzothiazole-6-sulfonyl)-isobutyl-amino]-1-benzyl-2-hydroxypropylcarbamoyl}-methoxy)-3,5-dimethyl-benzyl]-carbamic acid tert-butyl ester. This intermediate (500 mg) was further dissolved in methanol (20 ml) and 10 ml of a solution of HCl in isopropanol (5 to 6 N) was added dropwise. The mixture was stirred overnight at room temperature. The solvent was evaporated and the residue was purified on silica yielding 190 mg of compound 17 (43%).

### EXAMPLE 5

### Compound 27

A mixture of 134 mg of 2-methylamino-benzothiazole-6-sulfonic acid (3-amino-2-hydroxy-4-phenyl-butyl)-isobutyl amide, 4 mg of HOBT (hydroxybenzotriazol), 66 mg of EDC and 63 mg of 4-bromo-2-methyl benzoic acid in dichloromethane, was stirred overnight at room temperature. The reaction mixture was then washed with water and brine. The organic layer was separated, dried and the solvent evaporated. The residue was purified by preparative HPLC yielding 25 mg (13%) of compound 27.

### EXAMPLE 6

### Compound 28

To a mixture of 4.48 g 2-methylamino-benzothiazole-6-sulfonic acid (3-amino-2-hydroxy-4-phenyl-butyl)-isobutyl amide and 2.73 g triethylamine in dichloromethane was added 3.45 g carbonic acid 2,5-dioxo-pyrrolidin-1-yl ester oxazol-5-ylmethyl ester. This mixture was stirred at room temperature for 12 hours. After evaporation of dichloromethane under reduced pressure, the crude product was purified on silica, yielding 1.02 g 19% compound 28.

The compounds in Table 1, not intended to be limiting the scope of the present invention, have been prepared analogous to one of the above examples and tested in support of the presently claimed invention:

TABLE 1

| Name | Number |
|---|---|
| {(1S,2R)-3-[(2-amino-benzothiazole-6- | 1 |
| sulfonyl)-isobutyl-amino]-1-benzyl-2- | |
| hydroxypropyl}-carbamic acid hexahydro- | |
| furo[2,3-b]furan-3-yl ester | |
| {(1S,2R)-3-[(2-amino-benzothiazole-6- | 2 |
| sulfonyl)-isobutyl-amino]-1-benzyl-2- | |
| hydroxypropyl}-carbamic acid(3R,3aS,6aR)- | |
| hexahydro-furo[2,3-b]furan-3-yl ester | |
| {(1S,2R)-3-[(2-amino-benzothiazole-6- | 3 |
| sulfonyl)-isobutyl-amino]-1-benzyl-2- | |
| hydroxypropyl}-carbamic acid thiazol-5- | |
| ylmethyl ester | |
| {(1S,2R)-1-benzyl-2-hydroxy-3-[isobutyl- | 4 |
| (2-methylamino-benzothiazole-6- | |
| sulfonyl)-amino]-propyl}-carbamic acid | |
| hexahydro-furo[2,3-b]furan-3-yl ester | |
| {(1S,2R)-1-benzyl-2-hydroxy-3-[isobutyl- | 5 |
| (2-methylamino-benzothiazole-6- | |
| sulfonyl)-amino]-propyl}-carbamic | |
| acid(3R,3aS,6aR)-(hexahydro-furo[2,3- | |
| b]furan-3-yl)ester | |
| {(1S,2R)-1-benzyl-3-[(2-dimethylamino- | 6 |
| benzothiazole-6-sulfonyl)-isobutyl- | |
| amino]-2-hydroxy-propyl}-carbamic acid | |
| hexahydro-furo[2,3-b]furan-3-yl ester | |
| {(1S,2R)-3-[(2-amino-benzothiazole-6- | 7 |
| sulfonyl)-isobutyl-amino]-1-benzyl-2- | |
| hydroxypropyl}-carbamic acid benzyl ester | |
| N-{(1S,2R)-3-[(2-amino-benzothiazole- | 8 |
| 6-sulfonyl)-isobutyl-amino]-1-benzyl-2- | |
| hydroxypropyl}-2-(2,6-dimethyl-phenoxy)- | |
| acetamide | |
| {(1S,2R)-3-[(2-amino-benzothiazole-6- | 9 |
| sulfonyl)-isobutyl-amino]-1-benzyl-2- | |
| hydroxypropyl}-carbamic acid pyridin-3- | |
| ylmethyl ester | |
| 3-amino-N-{(1S,2R)-3-[(2-amino- | 10 |
| benzothiazole-6-sulfonyl)-isobutyl-amino]-1- | |
| benzyl-2-hydroxypropyl}-2-methyl-benzamide | |
| N-{(1S,2R)-3-[(2-amino-benzothiazole- | 11 |
| 6-sulfonyl)-isobutyl-amino]-1-benzyl-2- | |
| hydroxypropyl}-3-hydroxy-2-methyl-benzamide | |
| {(1S,2R)-3-[(2-amino-benzothiazole-6- | 12 |
| sulfonyl)-isobutyl-amino]-1-benzyl-2- | |
| hydroxypropyl}-carbamic acid tetrahydro- | |
| furan-3-yl ester | |
| {(1S,2R)-3-[(2-amino-benzothiazole-6- | 13 |
| sulfonyl)-isobutyl-amino]-1-benzyl-2- | |
| hydroxypropyl}-carbamic acid(S) | |
| (tetrahydro-furan-3-yl)ester | |
| N-{(1S,2R)-3-[(2-amino-benzothiazole- | 14 |
| 6-sulfonyl)-isobutyl-amino]-1-benzyl-2- | |
| hydroxypropyl}-2-methyl-benzamide | |
| N-{(1S,2R)-1-benzyl-2-hydroxy-3-[isobutyl- | 15 |
| (2-methylamino-benzothiazole-6- | |
| sulfonyl)-amino]-propyl}-2-(2,6- | |
| dimethyl-phenoxy)-acetamide; | |
| N-{(1S,2R)-3-[(2-amino-benzothiazole- | 16 |
| 6-sulfonyl)-isobutyl-amino]-1-benzyl-2- | |
| hydroxypropyl}-3-fluoro-2-methyl-benzamide | |
| N-{(1S,2R)-3-[(2-amino-benzothiazole- | 17 |
| 6-sulfonyl)-isobutyl-amino]-1-benzyl-2- | |
| hydroxypropyl}-2-(4-aminomethyl-2,6- | |
| dimethyl-phenoxy)-acetamide | |
| {(1S,2R)-1-benzyl-2-hydroxy-3-[isobutyl- | 18 |
| (2-methylamino-benzothiazole-6- | |
| sulfonyl)-amino]-propyl}-carbamic | |
| acid thiazol-5-ylmethyl ester | |
| {(1S,2R)-1-benzyl-2-hydroxy-3-[isobutyl- | 19 |
| (2-methylamino-benzothiazole-6- | |
| sulfonyl)-amino]-propyl}-carbamic | |
| acid thiazol-5-ylmethyl ester trifluoroacetate | |
| 3-amino-N-{(1S,2R)-1-benzyl-2-hydroxy- | 20 |
| 3-[isobutyl-(2-methylamino- | |
| benzothiazole-6-sulfonyl)-amino]- | |
| propyl}-2-methyl-benzamide trifluoroacetate | |
| {(1S,2R)-1-benzyl-2-hydroxy-3- | 21 |
| [isobutyl-(2-methylamino-benzothiazole-6- | |
| sulfonyl)-amino]-propyl}-carbamic | |
| acid(S)(tetrahydro-furan-3-yl)ester | |
| {(1S,2R)-1-benzyl-2-hydroxy-3- | 22 |
| [isobutyl-(2-methylamino-benzothiazole-6- | |
| sulfonyl)-amino]-propyl}-carbamic | |
| acid(S)(tetrahydro-furan-3-yl)ester | |
| trifluoroacetate | |
| N-{(1S,2R)-1-benzyl-2-hydroxy-3- | 23 |
| [isobutyl-(2-methylamino-benzothiazole-6- | |
| sulfonyl)-amino]-propyl}-3-hydroxy- | |
| 2-methyl-benzamide trifluoroacetate | |
| N-{(1S,2R)-3-[(2-amino-benzothiazole- | 24 |
| 6-sulfonyl)-isobutyl-amino]-1-benzyl-2- | |
| hydroxypropyl}-2-(4-iodo-2,6-dimethyl- | |
| phenoxy)-acetamide | |
| 2-(4-aminomethyl-2,6-dimethyl-phenoxy)- | 25 |
| N-{(1S,2R)-1-benzyl-2-hydroxy-3- | |
| [isobutyl-(2-methylamino-benzothiazole- | |
| 6-sulfonyl)-amino]-propyl}-acetamide | |
| 2-(4-amino-2,6-dimethyl-phenoxy)-N- | 26 |
| {(1S,2R)-1-benzyl-2-hydroxy-3-[isobutyl-(2- | |
| methylamino-benzothiazole-6-sulfonyl)- | |
| amino]-propyl}-acetamide | |
| N-{(1S,2R)-1-benzyl-2-hydroxy-3- | 27 |
| [isobutyl-(2-methylamino-benzothiazole-6- | |
| sulfonyl)-amino]-propyl}-4-bromo- | |
| 2-methyl-benzamide | |
| {(1S,2R)-1-benzyl-2-hydroxy-3- | 28 |
| [isobutyl-(2-methylamino-benzothiazole-6- | |
| sulfonyl)-amino]-propyl}-carbamic | |
| acid oxazol-5-ylmethyl ester | |
| 4-amino-N-{(1S,2R)-1-benzyl-2-hydroxy- | 29 |
| 3-[isobutyl-(2-methylamino- | |
| benzothiazole-6-sulfonyl)-amino]- | |
| propyl}-3-hydroxy-2-methyl-benzamide | |

Antiviral Analyses:

The compounds of the present invention were examined for anti-viral activity in a cellular assay. The assay demonstrated that these compounds exhibited potent anti-HIV activity against a wild type laboratory HIV strain (HIV-1 strain LAI). The cellular assay was performed according to the following procedure.

Cellular Assay Experimental Method:

HIV- or mock-infected MT4 cells were incubated for five days in the presence of various concentrations of the inhibitor. At the end of the incubation period, the replicating virus in the control cultures has killed all HIV-infected cells in the absence of any inhibitor. Cell viability is measured by measuring the concentration of MTT, a yellow, water soluble tetrazolium dye that is converted to a purple, water insoluble formazan in the mitochondria of living cells only. Upon solubilization of the resulting formazan crystals with isopropanol, the absorbance of the solution is monitored at 540 nm. The values correlate directly to the number of living cells remaining in the culture at the completion of the five-day incubation. The inhibitory activity of the compound was monitored on the virus-infected cells and was expressed as EC₅₀ and EC₉₀. These values represent the amount of the compound required to protect 50% and 90%, respectively, of the cells from the cytopathogenic effect of the virus. The toxicity of the compound was measured on the mock-infected cells and was expressed as CC₅₀, which represents the concentration of compound required to inhibit the growth of the cells by 50%. The selectivity index (SI) (ratio CC₅₀/EC₅₀) is an indication of the selectivity of the anti-HIV activity of the inhibitor. Wherever results are reported as e.g. pEC₅₀ or pCC₅₀ values, the result is expressed as the negative logarithm of the result expressed as EC₅₀ or CC₅₀ respectively.

Antiviral Spectrum:

Because of the increasing emergence of drug resistant HIV strains, the present compounds were tested for their potency against clinically isolated HIV strains harboring several mutations (Table 2 and 3). These mutations are associated with resistance to protease inhibitors and result in viruses that show various degrees of phenotypic cross-resistance to the currently commercially available drugs such as for instance saquinavir, ritonavir, nelfinavir, indinavir and amprenavir.

TABLE 2
List with a representative selection of mutant HIV strains (A to F).

| Strain | Mutations in HIV protease gene |
|---|---|
| A | V003I, L010I, V032T, L033M, E035D, S037Y, |
| | S037D, M046I, R057R/K, Q058E, L063P, |
| | K070T, A071V, I072V, I084V, L089V |
| B | V003I, L010I, K020R, E035D, M036I, S037N, |
| | Q058E, I062V, L063P, A071V, I072M, |
| | G073S, V077I, I084V, I085V, L090M |
| C | V003I, L010I, I015V, L019I, K020M, S037N, |
| | R041K, I054V, Q058E, L063P, A071V, |
| | I084V, L090M, I093L |
| D | V0031, L010L/I, I013V, L033I, E035D, M036I, |
| | M046L, K055R, R057K, L063P, I066F, |
| | A071V, I084V, N088D, L090M |
| E | V003I, L010I, V011I, A022V, L024I, E035D, |
| | M036I, S037T, R041K, I054V, I062V, |
| | L063P, A071V, I084V |
| F | L010F, M046I, M071V, I084V |

Results:

As a measure of the broad spectrum activity of the present compounds, Table 3 shows the results of the antiviral testing in terms of pEC₅₀. (=−log of EC₅₀). The fold resistance (FR), defined as FR=EC₅₀ (mutant strain)/EC₅₀ (HIV-1 strain LAI) is listed in Table 4. For most of the compounds the fold resistance ranges between 0.1 and 100. Thus, the present compounds are potent inhibitors of a broad range of mutant strains. The toxicity (Tox) is expressed as the pCC₅₀ value as determined with mock transfected cells while the pEC₅₀ for the wild type is displayed in column WT.

TABLE 3
Results of the toxicity testing and the resistance
testing against strain A to F (expressed as pEC₅₀).

| | Strain | Strain | Strain | Strain | Strain | Strain | | |
|---|---|---|---|---|---|---|---|---|
| Compound | A | B | C | D | E | F | Tox | WT |
| 1 | 8.53 | 8.44 | 8.38 | 8.65 | 8.51 | ND | 4.16 | 8.26 |
| 2 | 8.68 | 8.59 | 8.54 | 8.69 | 8.50 | 8.45 | 4.07 | 8.18 |
| 3 | 7.52 | 8.05 | 7.81 | 7.44 | 7.66 | 7.27 | 4.13 | 8.34 |
| 4 | 8.44 | 8.93 | 8.93 | 8.93 | 8.89 | 8.06 | <5 | 9.34 |
| 5 | 9.37 | 9.57 | 9.71 | ND | ND | 8.71 | 4.15 | 9.26 |
| 6 | 6.70 | 6.89 | 7.47 | 6.96 | ND | 6.15 | <4 | 8.4 |
| 7 | 6.37 | 7.53 | 7.49 | 6.93 | 7.36 | 6.11 | 4.33 | 8.23 |
| 8 | 7.5 | 7.87 | 7.59 | 7.47 | 7.56 | 6.85 | <5 | 8.18 |
| 9 | 6.58 | 8.25 | 5.31 | 7.38 | 7.62 | ND | 4.29 | 8.31 |
| 10 | 7.07 | 8.03 | 7.80 | 7.64 | 7.88 | 7.06 | 4.14 | 8.04 |
| 11 | 6.95 | 8.14 | 8.12 | 8.08 | 8.14 | 6.99 | 4.24 | 7.84 |
| 12 | 6.64 | 8.12 | 6.72 | 7.58 | 8.11 | ND | <4 | 8.37 |
| 13 | 7.39 | 8.24 | 8.42 | 8.13 | 8.57 | 6.98 | <4 | 8.52 |
| 14 | 6.05 | 7.57 | 6.75 | 7.40 | 7.52 | ND | 4.33 | 8.42 |
| 15 | 7.29 | 7.54 | 7.40 | 7.30 | 7.44 | 6.64 | 4.04 | 7.95 |
| 16 | <4 | <4 | <4 | <4 | <4 | <4 | 4.95 | 5.85 |
| 17 | 7.50 | 8.18 | 7.91 | 7.63 | 8.12 | 6.80 | 4.2 | 8.15 |
| 18 | 7.51 | 8.21 | 8.13 | 7.67 | 8.04 | 6.83 | 4.07 | 8.72 |
| 19 | 7.36 | 7.80 | 7.88 | 7.43 | 7.90 | 6.80 | ND | 8.51 |
| 20 | 6.50 | 7.61 | 7.40 | 7.38 | 7.59 | 6.12 | <4 | 8.32 |
| 22 | 6.93 | 7.83 | 8.19 | 7.78 | 8.36 | 6.1 | <4 | 8.84 |
| 23 | 6.54 | 8.02 | 8.06 | 7.67 | 8.14 | 5.20 | 4.16 | 8.34 |
| 25 | 7.36 | 7.70 | 7.75 | 7.39 | 7.76 | 6.32 | 4.85 | 8.39 |
| 26 | 7.52 | 8.40 | 8.14 | 8.08 | 8.21 | 7.25 | ND | 8.57 |
| 27 | 6.80 | 7.69 | 5.30 | 7.07 | 7.51 | 6.19 | <4 | 7.72 |
| 28 | 7.71 | 8.25 | 8.21 | 7.43 | 8.15 | 7.11 | ND | 8.60 |

ND means not determined

Some compounds have been tested for an even broader range of mutant HIV protease viruses. For instance, compound 1 was tested against a panel of more than 20 mutant proteases whereby compound 1 had a pIC₅₀ value of 9.13 for the most sensitive mutant and a pIC₅₀ value of 8.12 for the most resistant mutant. This indicates that all the mutants within this set of more that 20 mutant proteases are sensitive within a narrow window of IC₅₀ values and thus also in fold resistance values.

TABLE 4
fold resistance

| | Strain | Strain | Strain | Strain | Strain | Strain |
|---|---|---|---|---|---|---|
| Compound | A | B | C | D | E | F |
| 1 | 0.5 | 0.7 | 0.8 | 0.4 | 0.6 | — |
| 2 | 0.3 | 0.4 | 0.4 | 0.3 | 0.5 | 0.5 |
| 3 | 6.6 | 1.9 | 3.4 | 7.9 | 4.8 | 11.7 |
| 4 | 7.9 | 2.6 | 2.6 | 2.6 | 2.8 | 19.1 |
| 5 | 0.8 | 0.5 | 0.4 | — | — | 3.5 |
| 6 | 50.1 | 32.4 | 8.5 | 27.5 | — | 177.8 |
| 7 | 72.4 | 5.0 | 5.5 | 20.0 | 7.4 | 131.8 |
| 8 | 4.8 | 2.0 | 3.9 | 5.1 | 4.2 | 21.4 |
| 9 | 53.7 | 1.1 | 1000.0 | 8.5 | 4.9 | — |
| 10 | 9.3 | 1.0 | 1.7 | 2.5 | 1.4 | 9.5 |
| 11 | 7.8 | 0.5 | 0.5 | 0.6 | 0.5 | 7.1 |
| 12 | 53.7 | 1.8 | 44.7 | 6.2 | 1.8 | — |
| 13 | 13.5 | 1.9 | 1.3 | 2.5 | 0.9 | 34.7 |
| 14 | 234.4 | 7.1 | 46.8 | 10.5 | 7.9 | — |
| 15 | 4.6 | 2.6 | 3.5 | 4.5 | 3.2 | 20.4 |
| 17 | 4.5 | 0.9 | 1.7 | 3.3 | 1.1 | 22.4 |
| 18 | 16.2 | 3.2 | 3.9 | 11.2 | 4.8 | 77.6 |
| 19 | 14.1 | 5.1 | 4.3 | 12.0 | 4.1 | 51.3 |
| 20 | 66.1 | 5.1 | 8.3 | 8.7 | 5.4 | 158.5 |
| 22 | 81.3 | 10.2 | 4.5 | 11.5 | 3.0 | 549.5 |
| 23 | 63.1 | 2.1 | 1.9 | 4.7 | 1.6 | 1380.4 |
| 25 | 10.7 | 4.9 | 4.4 | 10.0 | 4.3 | 117.5 |
| 26 | 11.2 | 1.5 | 2.7 | 3.1 | 2.3 | 20.9 |
| 27 | 8.3 | 1.1 | 263.0 | 4.5 | 1.6 | 33.9 |
| 28 | 7.8 | 2.2 | 2.5 | 14.8 | 2.8 | 30.9 |

Protein Binding Analyses:

Human serum proteins like albumin (HSA) or alpha-1 acid glycoprotein (AAG) are known to bind many drugs, resulting in a possible decrease in the effectiveness of the drug. In order to determine whether the present compounds would be adversely effected by this binding, the anti-HIV activity of some of the present compounds was measured in the presence of human serum, thus evaluating the effect of the binding of the protease inhibitors to those proteins.

MT4 cells are infected with HIV-1 LAI at a multiplicity of infection (MOI) of 0.001-0.01 CCID₅₀ (50% cell culture infective dose per cell, CCID₅₀). After 1 h incubation, cells are washed and plated into a 96 well plate containing serial dilutions of the compound in the presence of 10% FCS (foetal calf serum), 10% FCS+1 mg/ml AAG (α₁-acid glycoprotein), 10% FCS+45 mg/ml HSA (human serum albumin) or 50% human serum (HS). After 5 or 6 days incubation, the EC₅₀ (50% effective concentration in cell-based assays) is calculated by determining the cell viability or by quantifying the level of HIV replication. Cell viability is measured using the assay described above. Into a 96 well plate containing serial dilutions of the compound in the presence of 10% FCS or 10% FCS+1 mg/ml AAG, HIV (wild type or resistant strain) and MT4 cells are added to a final concentration of 200-250 CCID₅₀/well and 30,000 cells/well, respectively. After 5 days of incubation (37° C., 5% CO₂), the viability of the cells is determined by the tetrazolium colorimetric MTT (3-[4,5-Dimethylthiazol-2-yl]-2,5-diphenyltetrazolium bromide) method (Pauwels et al. J Virol. Methods 1988, 20, 309321).

Pharmacokinetic Data

The pharmacokinetic properties of some compounds of formula (I) were tested on rats and dogs. The compounds were evaluated in Whistar rats, source Iffa Credo, weighing approximately 350 g. Before dosing the animals were fasted overnight (approximately 12 h fasting period). The compounds were dissolved in DMSO or PEG 400. The results represented in the table concern the results from the oral or intra-peritoneal dosing of the compounds. Blood was sampled at 30 min, 1 h, 2 h, 3 h, no pre-dose sample was taken. The amount of the compound in the biological sample was determined using LC-MS. In the table below “or” means oral dosing, “ip” means intraperitoneal dosing, “mpk” means mg per kilogram. The results are illustrated in Table 5.

TABLE 5

| | | conditions |
|---|---|---|
| Compound | Cₘₐₓ (ng/ml) | (results normalized to 10 mpk) |
| 2 | 427 after 30 minutes | ip, rat, DMSO |
| 2 | 52 after 30 minutes | or, rat, PEG400 |
| 4 | 1668 after 30 minutes | ip, rat, DMSO |
| 4 | 348 after 30 minutes | or, rat, DMSO |
| 4 | 225 after 30 minutes | or, rat, PEG400 |
| 15 | 86 after 30 minutes | ip, rat, DMSO |
| 15 | 10 after 180 minutes | or, rat, PEG400 |
| 18 | 1141 after 240 minutes | ip, rat, DMSO |
| 18 | 396 after 30 minutes | or, rat, DMSO |
| 18 | 668 after 15 minutes | or, rat, PEG400 |
| 18 | 15 after 60 minutes | or, dog, DMSO |
| 18 | 42 after 30 minutes | or, dog, PEG400 |
| 21 | 1763 after 15 minutes | ip, rat, DMSO |
| 21 | 1139 after 15 minutes | or, rat, DMSO |
| 21 | 1315 after 15 minutes | or, rat, PEG400 |
| 21 | 61 after 120 minutes | or, dog, PEG400 - 2 animals |
| 21 | 184 after 30 minutes | or, dog, PEG400 - 4 animals |
| 25 | 453 after 30 minutes | ip, rat, DMSO |
| 28 | 1003 after 30 minutes | ip, rat, DMSO |
| 28 | 540 after 30 minutes | or, rat, DMSO |
| 28 | 430 after 60 minutes | or, rat, PEG400 |

Formulation

Active ingredient, in casu a compound of formula (I), is dissolved in organic solvent such as ethanol, methanol or methylene chloride, preferably, a mixture of ethanol and methylene chloride. Polymers such as polyvinylpyrrolidone copolymer with vinyl acetate (PVP-VA) or hydroxypropylmethylcellulose (HPMC), typically 5 mPa.s, are dissolved in organic solvents such as ethanol, methanol methylene chloride. Suitably the polymer is dissolved in ethanol. The polymer and compound solutions are mixed and subsequently spray dried. The ratio of compound/polymer can be selected from 1/1 to 1/6. Intermediate ranges were 1/1.5 and 1/3. The spraydried powder, a solid dispersion, is subsequently filled in capsules for administration. The drug load in one capsule depends on the capule size used.

Film-Coated Tablets

Preparation of Tablet Core

A mixture of 100 g of active ingredient, in casu a compound of formula (I), 570 g lactose and 200 g starch was mixed well and thereafter humidified with a solution of 5 g sodium dodecyl sulfate and 10 g polyvinylpyrrolidone in about 200 ml of water. The wet powder mixture was sieved, dried and sieved again. Then there was added 100 g microcrystalline cellulose and 15 g hydrogenated vegetable oil. The whole was mixed well and compressed into tablets, giving 10,000 tablets, each comprising 10 mg of the active ingredient.

Coating

To a solution of 10 g methylcellulose in 75 ml of denaturated ethanol there was added a solution of 5 g of ethylcellulose in 150 ml of dichloromethane. Then there were added 75 ml of dichloromethane and 2.5 ml 1,2,3-propanetriol. 10 g of polyethylene glycol was molten and dissolved in 75 ml of dichloromethane. The latter solution was added to the former and then there were added 2.5 g of magnesium octadecanoate, 5 g of polyvinylpyrrolidone and 30 ml of concentrated color suspension and the whole was homogenated. The tablet cores were coated with the thus obtained mixture in a coating apparatus.

1. A method of inhibiting mutant HIV protease in a mammal infected with said mutant HIV protease, said method comprising the step of administering to said mammal a therapeutically effective amount of a compound having the formula 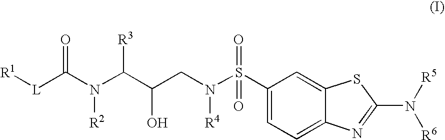
a N-oxide, salt, stereoisomeric form, racemic mixture, prodrug, ester or metabolite thereof, wherein
R₁ is hexahydrofuro[2,3-b]furanyl, tetrahydrofuranyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino;
R₂ is hydrogen or C₁₋₆alkyl;
L is a direct bond, —O—, C₁₋₆alkanediyl-O— or —O—C₁₋₆alkanediyl;
R₃ is phenylC₁₋₄alkyl;
R₄ is C₁₋₆alkyl;
R₅ is hydrogen or C₁₋₆alkyl;
R₆ is hydrogen or C₁₋₆alkyl.

2. The method according to claim 1 wherein
R² is hydrogen;
R³ is phenylmethyl;
R⁴ is C₁₋₄alkyl, preferably isobutyl;
R⁵ is hydrogen or methyl;
R⁶ is hydrogen or methyl.

3. The method according to claim 1 wherein R⁵ is methyl or hydrogen and R⁶ is hydrogen

4. The method according to claim 1 wherein both R⁵ and R⁶ are hydrogen.

5. The method according to claim 1 wherein -L-R¹ is —O-hexahydrofuro[2,3-b]furanyl), —O-tetrahydrofuranyl, —O-methyl-(optionally substituted phenyl), —O-methyl-pyridinyl, —O-methyl-thiazolyl, —O-methyl-thiazolyl, -methyl-O-(optionally substituted phenyl) or optionally substituted phenyl.

6. A method of inhibiting mutant HIV protease in a mammal infected with said mutant HIV protease, said method comprising the step of administering to said mammal a therapeutically effective amount of a compound selected from the group consisting of:
{3-[(2-amino-benzothiazole-6-sulfonyl)-isobutyl-amino]-1-benzyl-2-hydroxypropyl}-carbamic acid hexahydro-furo[2,3-b]furan-3-yl ester;
{3-[(2-amino-benzothiazole-6-sulfonyl)-isobutyl-amino]-1-benzyl-2-hydroxypropyl}-carbamic acid thiazol-5-ylmethyl ester;
{1-benzyl-2-hydroxy-3-[isobutyl-(2-methylamino-benzothiazole-6-sulfonyl)-amino]-propyl}-carbamic acid hexahydro-furo[2,3-b]furan-3-yl ester;
{1-benzyl-3-[(2-dimethylamino-benzothiazole-6-sulfonyl)-isobutyl-amino]-2-hydroxypropyl}-carbamic acid hexahydro-furo[2,3-b]furan-3-yl ester;
{3-[(2-amino-benzothiazole-6-sulfonyl)-isobutyl-amino]-1-benzyl-2-hydroxypropyl}-carbamic acid benzyl ester;
N-{3-[(2-amino-benzothiazole-6-sulfonyl)-isobutyl-amino]-1-benzyl-2-hydroxypropyl}-2-(2,6-dimethyl-phenoxy)-acetamide;
{3-[(2-amino-benzothiazole-6-sulfonyl)-isobutyl-amino]-1-benzyl-2-hydroxypropyl}-carbamic acid pyridin-3-ylmethyl ester;
3-amino-N-{3-[(2-amino-benzothiazole-6-isobutyl-amino]-1-benzyl-2-hydroxypropyl}-2-methyl-benzamide;
N-{3-[(2-amino-benzothiazole-6-sulfonyl)-isobutyl-amino]-1-benzyl-2-hydroxypropyl}-3-hydroxy-2-methyl-benzamide;
{3-[(2-amino-benzothiazole-6-sulfonyl)-isobutyl-amino]-1-benzyl-2-hydroxypropyl}-carbamic acid tetrahydro-furan-3-yl ester;
N-{3-[(2-amino-benzothiazole-6-sulfonyl)-isobutyl-amino]-1-benzyl-2-hydroxypropyl}-2-methyl-benzamide;
N-{1-benzyl-2-hydroxy-3-[isobutyl-(2-methylamino-benzothiazole-6-sulfonyl)-amino]-propyl}-2-(2,6-dimethyl-phenoxy)-acetamide;
N-{3-[(2-amino-benzothiazole-6-sulfonyl)-isobutyl-amino]-1-benzyl-2-hydroxypropyl}-3-fluoro-2-methyl-benzamide;
N-{3-[(2-amino-benzothiazole-6-sulfonyl)-isobutyl-amino]-1-benzyl-2-hydroxypropyl}-2-(4-aminomethyl-2,6-dimethyl-phenoxy)-acetamide;
{1-benzyl-2-hydroxy-3-[isobutyl-(2-methylamino-benzothiazole-6-sulfonyl)-amino]-propyl}-carbamic acid thiazol-5-ylmethyl ester;
3-amino-N-{1-benzyl-2-hydroxy-3-[isobutyl-(2-methylamino-benzothiazole-6-sulfonyl)-amino]-propyl}-2-methyl-benzamide;
{1-benzyl-2-hydroxy-3-[isobutyl-(2-methylamino-benzothiazole-6-sulfonyl)-amino]-propyl}-carbamic acid tetrahydro-furan-3-yl ester;
N-{1-benzyl-2-hydroxy-3-[isobutyl-(2-methylamino-benzothiazole-6-sulfonyl)-amino]-propyl}-3-hydroxy-2-methyl-benzamide;
N-{3-[(2-amino-benzothiazole-6-sulfonyl)-isobutyl-amino]-1-benzyl-2-hydroxypropyl}-2-(4-iodo-2,6-dimethyl-phenoxy)-acetamide;
2-(4-aminomethyl-2,6-dimethyl-phenoxy)-N-{1-benzyl-2-hydroxy-3-[isobutyl-(2-methylamino-benzothiazole-6-sulfonyl)-amino]-propyl}-acetamide;
2-(4-amino-2,6-dimethyl-phenoxy)-N-{1-benzyl-2-hydroxy-3-[isobutyl-(2-methylamino-benzothiazole-6-sulfonyl)-amino]-propyl}-acetamide;
N-{1-benzyl-2-hydroxy-3-[isobutyl-(2-methylamino-benzothiazole-6-sulfonyl)-amino]-propyl}-4-bromo-2-methyl-benzamide;
{1-benzyl-2-hydroxy-3-[isobutyl-(2-methylamino-benzothiazole-6-sulfonyl)-amino]-propyl}-carbamic acid oxazol-5-ylmethyl ester;
4-amino-N-{1-benzyl-2-hydroxy-3-[isobutyl-(2-methylamino-benzothiazole-6-sulfonyl)-amino]-propyl}-3-hydroxy-2-methyl-benzamide; and
or a salt, or a stereoisomeric form thereof.

7. The method according to claim 1 wherein the mutant HIV protease has at least one mutation at a position selected from 10, 71 and 84.

8. The method according to claim 1 wherein the fold resistance of the mutant HIV protease for the compound described in claim 1 ranges between 0.01 and 100.

9. A compound having the formula 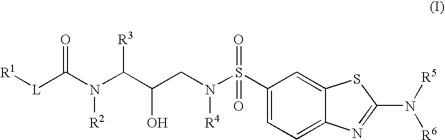
a N-oxide, salt, stereoisomeric form, racemic mixture, prodrug, ester or metabolite thereof, wherein
R₁ is hexahydrofuro[2,3-b]furanyl, tetrahydrofuranyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino;
R₂ is hydrogen or C₁₋₆alkyl;
L is a direct bond, —O—, C₁₋₆alkanediyl-O— or —O—C₁₋₆alkanediyl;
R₃ is phenylC₁₋₄alkyl;
R₄ is C₁₋₆alkyl;
R₅ is hydrogen or C₁₋₆alkyl;
R₆ is hydrogen or C₁₋₆alkyl;
provided that the compound is other than:
{(1S,2R)-3-[(2-amino-benzothiazole-6-sulfonyl)-isobutyl-amino]-1-benzyl-2-hydroxypropyl}-carbamic acid benzyl ester;
{(1S,2R)-3-[(2-amino-benzothiazole-6-sulfonyl)-isobutyl-amino]-1-benzyl-2-hydroxypropyl}-carbamic acid pyridin-3-ylmethyl ester;
{(1S,2R)-3-[(2-amino-benzothiazole-6-sulfonyl)-isobutyl-amino]-1-benzyl-2-hydroxypropyl}-carbamic acid thiazol-5-ylmethyl ester;
{(1S,2R)-3-[(2-amino-benzothiazole-6-sulfonyl)-isobutyl-amino]-1-benzyl-2-hydroxypropyl}-2-(2,6-dimethyl-phenoxy)-acetamide;
3-amino-{(1S,2R)-3-[(2-amino-benzothiazole-6-sulfonyl)-isobutyl-amino]-1-benzyl-2-hydroxypropyl}-2-methyl-benzamide;
4-amino-{(1S,2R)-3-[(2-amino-benzothiazole-6-sulfonyl)-isobutyl-amino]-1-benzyl-2-hydroxypropyl}-2-methyl-benzamide;
5-amino-{(1S,2R)-3-[(2-amino-benzothiazole-6-sulfonyl)-isobutyl-amino]-1-benzyl-2-hydroxypropyl}-2-methyl-benzamide;
N-{(1S,2R)-3-[(2-amino-benzothiazole-6-sulfonyl)-isobutyl-amino]-1-benzyl-2-hydroxypropyl}-2-methyl-benzamide;
N-{(1S,2R)-3-[(2-amino-benzothiazole-6-sulfonyl)-isobutyl-amino]-1-benzyl-2-hydroxypropyl}-4-hydroxy-2-methyl-benzamide;
N-{(1S,2R)-3-[(2-amino-benzothiazole-6-sulfonyl)-isobutyl-amino]-1-benzyl-2-hydroxypropyl}-3-hydroxy-2-methyl-benzamide; and
{(1S,2R)-3-[(2-amino-benzothiazole-6-sulfonyl)-isobutyl-amino]-1-benzyl-2-hydroxypropyl}-carbamic acid (S)-(tetrahydrofuran-3-yl) ester.

10. A compound according to claim 9 wherein R¹ is hexahydrofuro[2,3-b]furanyl or oxazolyl.

11. A compound according to claim 9 wherein R₁ is hexahydrofuro[2,3-b]furanyl, tetrahydrofuranyl, oxazolyl, thiazolyl, and L is a direct bond.

12. A compound according to claim 9 wherein R₁ is hexahydrofuro[2,3-b]furanyl, oxazolyl, thiazolyl, pyridinyl, or phenyl optionally substituted with one or more substituents independently selected from C₁₋₆alkyl, hydroxy, amino, halogen, aminoC₁₋₄alkyl and mono- or di(C₁₋₄alkyl)amino; and L is —O—.

13. A compound according to claim 9 wherein wherein R₁ is hexahydrofuro[2,3-b]furanyl, tetrahydrofuranyl, oxazolyl, or phenyl substituted with one or more substituents independently selected from C₁₋₆alkyl, hydroxy, amino, halogen, aminoC₁₋₄alkyl and mono- or di(C₁₋₄alkyl)amino; and L is C₁₋₆alkanediyl-O— whereby the —O— is attached to the nitrogen of the amide.

14. A compound according to claim 9 wherein R₁ is hexahydrofuro[2,3-b]furanyl, tetrahydrofuranyl, oxazolyl, thiazolyl, pyridinyl, or phenyl optionally substituted with one or more substituents independently selected from hydroxy, amino, halogen, aminoC₁₋₄alkyl and mono- or di(C₁₋₄alkyl)amino; and L is —O—C₁₋₆alkanediyl whereby —O— is attached to the R¹ group.

15. A compound according to claim 9 wherein at least one of R₅ and R₆ is C₁₋₆alkyl.

16. A compound according to claim 9 wherein R² is hydrogen; R³ is phenylmethyl; R⁴ is C₁₋₄alkyl.

17. A compound according to claim 9 having the formula
{1-benzyl-2-hydroxy-3-[isobutyl-(2-methylamino-benzothiazole-6-sulfonyl)-amino]-propyl}-carbamic acid hexahydro-furo[2,3-b]furan-3-yl ester;
{1-benzyl-3-[(2-dimethylamino-benzothiazole-6-sulfonyl)-isobutyl-amino]-2-hydroxypropyl}-carbamic acid hexahydro-furo[2,3-b]furan-3-yl ester;
N-{1-benzyl-2-hydroxy-3-[isobutyl-(2-methylamino-benzothiazole-6-sulfonyl)-amino]-propyl}-2-(2,6-dimethyl-phenoxy)-acetamide;
N-{3-[(2-amino-benzothiazole-6-sulfonyl)-isobutyl-amino]-1-benzyl-2-hydroxypropyl}-3-fluoro-2-methyl-benzamide;
N-{3-[(2-amino-benzothiazole-6-sulfonyl)-isobutyl-amino]-1-benzyl-2-hydroxypropyl}-2-(4-aminomethyl-2,6-dimethyl-phenoxy)-acetamide;
{1-benzyl-2-hydroxy-3-[isobutyl-(2-methylamino-benzothiazole-6-sulfonyl)-amino]-propyl}-carbamic acid thiazol-5-ylmethyl ester;
3-amino-N-{1-benzyl-2-hydroxy-3-[isobutyl-(2-methylamino-benzothiazole-6-sulfonyl)-amino]-propyl}-2-methyl-benzamide;
{1-benzyl-2-hydroxy-3-[isobutyl-(2-methylamino-benzothiazole-6-sulfonyl)-amino]-propyl}-carbamic acid tetrahydro-furan-3-yl ester;
N-{1-benzyl-2-hydroxy-3-[isobutyl-(2-methylamino-benzothiazole-6-sulfonyl)-amino]-propyl}-3-hydroxy-2-methyl-benzamide;
N-{3-[(2-amino-benzothiazole-6-sulfonyl)-isobutyl-amino]-1-benzyl-2-hydroxypropyl}-2-(4-iodo-2,6-dimethyl-phenoxy)-acetamide;
2-(4-aminomethyl-2,6-dimethyl-phenoxy)-N-{1-benzyl-2-hydroxy-3-[isobutyl-(2-methylamino-benzothiazole-6-sulfonyl)-amino]-propyl}-acetamide;
2-(4-amino-2,6-dimethyl-phenoxy)-N-{1-benzyl-2-hydroxy-3-[isobutyl-(2-methylamino-benzothiazole-6-sulfonyl)-amino]-propyl}-acetamide;
N-{1-benzyl-2-hydroxy-3-[isobutyl-(2-methylamino-benzothiazole-6-sulfonyl)-amino]-propyl}-4-bromo-2-methyl-benzamide;
{1-benzyl-2-hydroxy-3-[isobutyl-(2-methylamino-benzothiazole-6-sulfonyl)-amino]-propyl}-carbamic acid oxazol-5-ylmethyl ester;
4-amino-N-{1-benzyl-2-hydroxy-3-[isobutyl-(2-methylamino-benzothiazole-6-sulfonyl)-amino]-propyl}-3-hydroxy-2-methyl-benzamide; or
a salt thereof, or a stereoisomeric form thereof.

18. A compound according to claim 9, selected from the group consisting of:
{3-[(2-amino-benzothiazole-6-sulfonyl)-isobutyl-amino]-1-benzyl-2-hydroxypropyl}-carbamic acid hexahydro-furo[2,3-b]furan-3-yl ester;
{1-benzyl-2-hydroxy-3-[isobutyl-(2-methylamino-benzothiazole-6-sulfonyl)-amino]-propyl}-carbamic acid hexahydro-furo[2,3-b]furan-3-yl ester;
{1-benzyl-3-[(2-dimethylamino-benzothiazole-6-sulfonyl)-isobutyl-amino]-2-hydroxypropyl}-carbamic acid hexahydro-furo[2,3-b]furan-3-yl ester;
or a salt or stereoisomeric form thereof.
